(19) United States
(12) Patent Application Publication
(10) Pub. No.: US 2013/0189245 A1
(43) Pub. Date: Jul. 25, 2013

(54) METHOD FOR PRODUCING TOXOIDS USING ALPHA-DICARBONYL COMPOUNDS

(71) Applicants: Lucy Jane Cork, Salisbury (GB); Gary John Phillips, Salisbury (GB); Gareth David Griffiths, Salisbury (GB); David Whitfield, Salisbury (GB)

(72) Inventors: Lucy Jane Cork, Salisbury (GB); Gary John Phillips, Salisbury (GB); Gareth David Griffiths, Salisbury (GB); David Whitfield, Salisbury (GB)

(21) Appl. No.: 13/810,520

(22) PCT Filed: Jul. 18, 2011
(86) PCT No.: PCT/GB11/01074

§ 371 Date: Apr. 11, 2013

(30) Foreign Application Priority Data
Jul. 16, 2010 (GB) 1011968.3

(51) Int. Cl. C07K 14/415; C07K 14/33

(52) U.S. Cl. CPC C07K 14/415; C07K 14/33
USPC 424/130.1; 530/370; 530/350; 424/184.1; 530/387.1

(57) ABSTRACT

The present invention relates to the use of toxoids prepared using a-dicarbonyl toxoiding reagents such as glyoxal, butanedione and phenylglyoxal. The toxoids may be prepared with low concentrations of toxoiding reagent and in short periods of time, often in as few as 24 hours, making the toxoiding reagents particularly advantageous when compared with traditional formaldehyde toxoiding. Toxoids prepared using dicarbonyl reagents such as phenylglyoxal are described and claimed as are pharmaceutical and vaccine compositions comprising the toxoids, methods of treatment using such compositions and antibodies generated by immunisation with the toxoid and methods of treatment using the antibodies so prepared or fragments of such antibodies.

This invention relates to toxoids and methods for producing toxoids from toxins. The toxoids of the invention are particularly useful in generating a therapeutic or prophylactic treatment for intoxication, and for producing therapeutic agents comprising antibodies generated by vaccination, using the toxoids. Toxoid vaccines and anti-toxoid antibody treatments, or antitoxins, form further aspects of the invention.

Toxins are poisonous substances produced by living cells or organisms and although toxins are traditionally considered to be toxic polypeptide or protein products of plants, animals, micro-organisms (including, but not limited to, bacteria, viruses, fungi, rickettsiae or protozoa), the term toxin also includes recombinant or synthesized molecules that mimic such toxic polypeptides and protein, irrespective of origin and method of production. Toxins usually cause disease on contact with or absorption by body tissues, whereupon the toxin acts as an antigen, and interacts with enzymes, cellular receptors and the like. Toxins vary greatly in their severity, ranging from usually minor and acute (e.g. a bee sting) to almost immediately deadly (e.g. botulinum and ricin toxins). The venom of many snakes contains powerful toxins. The severity of some toxins, coupled with comparatively simple methods of extraction or production has increased concern that toxins could be used by terrorists or military aggressors.

Toxoids are inactivated toxins. That is, toxins in which toxicity has been destroyed but which retain the property of inducing an immune response to the toxin, for example by producing an antibody response in a host animal. For this reason, toxoids are often used as vaccines to protect mammals against the effects of toxins and also in the preparation of antitoxins, i.e. antibodies or antibody products produced from the immune response to the toxoid.

Toxoids are commonly produced by chemical inactivation of the toxin, for example by direct chemical reaction of individual amino acid residues within the toxin. Toxoids of several toxins have been prepared using formaldehyde, which is known to react principally with lysine residues in the toxin protein. Both ricin and botulinum toxins have been toxoided (inactivated) successfully by reaction with formaldehyde and current vaccines and antitoxin therapies against these toxins are produced using formaldehyde inactivation.

However, toxoiding with formaldehyde is not a trivial procedure. Toxins are typically dialysed against low concentrations (0.2-0.6%) of formaldehyde at raised temperatures (typically 30-37° C.) for extended periods of time (usually 7 days or longer). For example, formaldehyde inactivation of ricin is typically done by incubating ricin (1 mg/ml) with formaldehyde (37% v/v) at 37° C. for 21 days. Formaldehyde inactivation is therefore time-consuming and costly in terms of manufacture (beyond laboratory-scale).

Additionally, the reaction of formaldehyde with toxin is complex and it is often difficult to obtain complete removal of toxicity. Consequently, toxoids produced with formaldehyde have undesirable properties in that the toxoid may revert to active toxin if stored inappropriately. This problem has been overcome in commercially available diphtheria and tetanus based toxoid vaccines by the inclusion of a small amount of formaldehyde in the final composition. However this is undesirable because formaldehyde has been classed as a probable human carcinogen.

It is also common that formaldehyde inactivation produces major conformational changes of the toxin, which makes the toxoid less immunologically similar to the toxin and lacking certain neutralising epitopes, which ultimately affects the ability of the toxoid to produce useful antibodies.

Alternatives to formaldehyde are known, as described for example in International patent Application no PCT/GB2006/000466 (published as WO 2006/085088), which describes the use of iodoacetamide to inactivate botulinum toxin. Iodoacetamide is a powerful alkylating agent and, whilst it has the ability to irreversibly alkylate cysteine residues in the toxin, it has also been identified as a suspected carcinogen and is known to be a light sensitive compound, which might limit its use in large-scale production of toxoid.

There remains a need, therefore, to develop toxoiding reagents which overcome the deficiencies of existing toxoiding reagents. Such a toxoiding reagent would, ideally, be safe and stable to handle and have the ability to completely inactivate toxins, without the risk of reversion to its toxic form, such that residual amounts of the toxoiding reagent (or other reagent such as formaldehyde) need not be included in any pharmaceutical compositions of the toxoid. The length of time taken to completely inactivate the toxin would ideally be much less than the time required for toxoiding with formaldehyde, and have the potential to be conducted at room temperature and without the need for harsh reaction conditions.

The present inventors have surprisingly found that toxins, and in particular protein toxins, may be completely inactivated by reacting the toxin with a suitable quantity of an α-dicarbonyl compound with general structure R—C(O)C(O)R′. This method of preparing a toxoid is rapid, with toxoids being produced in hours rather than days. Conveniently a toxoid may be prepared in 24 hours or less. Relatively small quantities of the toxoiding reagent are required, such that reactions may be effected using lower concentrations than are used with the traditional formaldehyde toxoiding. Toxoids produced using α-dicarbonyl aldehydes of general formula R—C(O)C(O)H (i.e. wherein R′ is hydrogen) are particularly effective and, phenylglyoxal, as an example of such an α-dicarbonyl aldehyde toxoiding reagent, are stable, immunogenic and appear to have a comparable (i.e. substantially unaltered) secondary structure to the toxin. The toxoids of the present invention, and exemplified herein, may be used as vaccines for the prophylactic or therapeutic treatment of intoxication by the toxin from which the toxoid is derived and are particularly useful in the production of therapeutic antitoxins, which may, in turn, be used to treat intoxication.

Accordingly, in a first aspect, the present invention provides a toxoid, derived from a toxin wherein arginine residues within the toxin have undergone chemical reaction with an α-dicarbonyl compound of general structure R—C(O)C(O)R′, as shown below

[RC(O)C(O)R′] wherein R, R′=H, alkyl, substituted alkyl, aryl or substituted aryl group

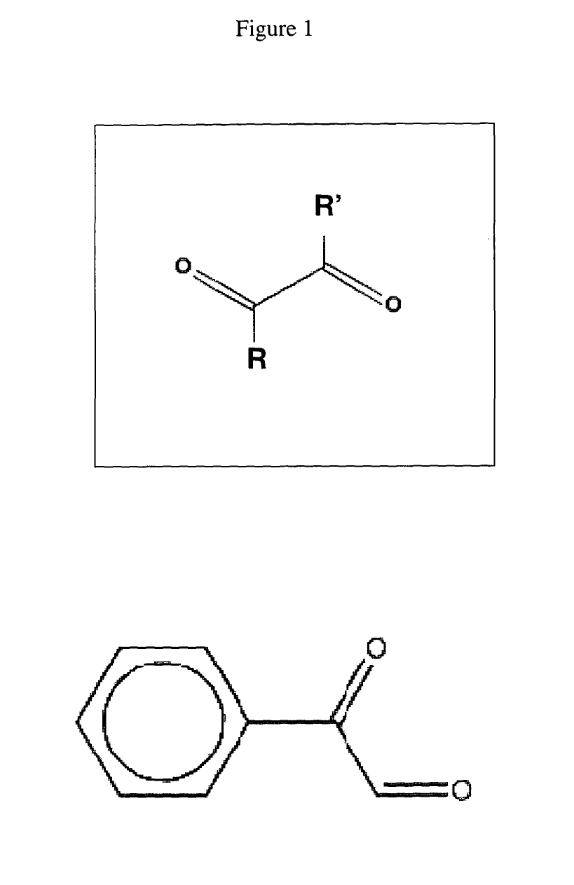

Structure A (see FIG. 1)

The dicarbonyl compound toxoiding reagent can be any possible chemical compound falling within the general structure above. For example, R or R′ in structure A may be Hydrogen or any alkyl or aryl group, such as methyl, ethyl, propyl, butyl, pentyl, cyclopentyl, hexyl, cyclohexyl, phenyl, etc (or branched or substituted variants of these). Accordingly the toxoiding reagent may be conveniently selected from the group consisting of glyoxal, methylglyoxal, butanedione, 1,2-cyclohexanedione, phenylglyoxal, 4-fluorophenylglyoxal, 4-nitrophenyl glyoxal and 4-hydroxy phenylglyoxal, all of which are currently commercially available.

Preferred embodiments of the invention are provided when R′ is hydrogen, such that the toxoiding agents fall within the general class of ketoaldehydes. A particularly preferred dicarbonyl (ketaldehyde) compound is phenylglyoxal (FIG. 1). Phenylglyoxal has been shown previously to react selectively with arginyl residues of proteins to give a product that contains two phenylglyoxal groups per guanidine group (of arginine) but the present inventors have surprisingly found that phenylglyoxal has high specificity for arginyl residues such that the toxoids produced are completely and irreversibly inactivated and are highly immunogenic, without the conformational structure of the toxin/toxoid being significantly altered.

Without being bound by theory, it is thought that the toxoiding reaction follows two steps. The first step is expected to be reversible condensation of one guanidine amino group with the least-hindered carbon atom of the glyoxal, i.e. CHO group in phenyl or 4-hydroxyphenyl glyoxal, or either of the two identical C═O groups in butanedione or cyclohexanedione. The second step, expected to be not easily reversible, is a second condensation of the remaining guanidine amino group to produce the heterocycle.

Phenylglyoxal (PG) is a good reagent for such a transformation as the electronegative phenylcarbonyl group will activate the less hindered CHO group to nucleophilic attack and facilitate the subsequent cyclisation. Consequently derivatives of phenylglyoxal, such as 4-hydroxyphenylglyoxal, may also be used to produce toxoids. 4-hydroxyphenylglyoxal may be less reactive that phenylglyoxal but might be advantageous as it is more water soluble. Other derivatives of PG with an electron-withdrawing group in the 4-position, such as the 4-cyano, -fluoro, -trifluoromethyl or—nitro derivatives should react even faster than PG and have the potential to produce toxoids in minutes.

In general, dicarbonyl toxoiding reagents with aldehyde groups (CHO) i.e. of general structure R—C(O)C(O)H will react more rapidly than those with ketone groups (C═O) i.e. of general structure R—C(O)C(O)R′ and, consequently, are preferred.

The dicarbonyl toxoiding reagent of the present invention, and in particular phenylglyoxal, may be used to produce toxoids of toxins derived from any source. For example, protein toxoids may be produced from toxins which are derived from plants. Suitable plant toxins include abrin and ricin. As shown hereinafter the toxoiding reagents are particularly useful for preparing toxoids of ricin.

It will be understood by the person skilled in the art, however, that the toxin may equally be derived from an animal or a micro-organism.

Particular animal toxins include, but are not limited to, snake toxins such as alpha-bungarotoxin, beta-bungarotoxin, cobratoxin, crotoxin, erabutoxin, taicatoxin and textilotoxin, spider toxins such as agatoxin, atracotoxin, grammotoxin, latrotoxin, phoneutriatoxin, phrixotoxin and versutoxin, and scorpion toxins such as margatoxin, iberiotoxin or noxiustoxin.

Toxins produced by micro-organisms and, in particular, exotoxins excreted by bacteria, fungi, algae, and protozoa may be toxoided and used as vaccines in the context of the present invention. Toxins produced by bacteria are of particular clinical significance as these toxins are a major cause of illness in humans, with the severity of infection ranging from lethal to the individual to being an underlying cause of diarrhoeal disease, which in turn represents a major health problem in developing countries. Accordingly, the toxoiding reagent and methods of the present invention are particularly useful in preparing toxoids of bacterial toxins and for their use as medicaments and for the preparation of antitoxins. Particularly useful examples of toxoids are those produced from Clostridial neurotoxins, such as botulinum toxin or tetanus toxin, diphtheria toxin, cholera toxin, Bordetella pertussis toxin, pseudomonas endotoxin A, shiga toxin or shiga-like toxins, E. coli heat labile toxin, anthrax toxin, SEB. The shiga-like toxin may be one produced by E. coli. The bacteria from which the toxin is derived is typically one involved in causing disease, such as Clostridium tetani, Clostridium botulinum, Corynebacterium diphtheriae, Vibrio cholerae, Shigella dysenteriae, Bordetella pertussis or Pseudomonas aeruginosa. As shown hereinafter the toxoiding reagents are particularly useful for preparing toxoids of clinical importance, such as C. difficile, cholera and diphtheria.

The toxoiding reagent and methods described herein are particularly useful in preparing toxoids from toxins that may be used as biological warfare or bio-terrorism agents. The toxoids of the invention may then be used directly as vaccines or may be used to generate an antibody response, which antibodies, once harvested, form the basis of a therapeutic treatment for intoxication by the toxin from which the toxoid is derived.

Consequently, in a second aspect, the invention provides a toxoid derived from a toxin which has undergone chemical reaction with an a-dicarbonyl compound of general structure R—C(O)C(O)R′ or R—C(O)C(O)H for use in the prophylactic or therapeutic treatment of intoxication by the toxin (toxin poisoning).

The α-dicarbonyl compound may be any of the compounds described above which are useful for preparing a toxoid. Particularly suitable dicarbonyl compounds include, but are not limited to, those selected from the group consisting of glyoxal, methylglyoxal, butanedione, 1,2-cyclohexanedione, 4-fluorophenylglyoxal, 4-nitrophenyl glyoxal, 4-hydroxy phenylglyoxal and phenylglyoxal.

In a particular embodiment, phenylglyoxal is used to prepare a toxoid of a particular toxin, which may isolated from the reaction mixture, purified if necessary and administered directly as a prophylactic or therapeutic vaccine against the toxin from which the toxoid is derived. Alternatively the toxoid may be formulated into a pharmaceutically acceptable formulation, with suitable diluents, excipients, carriers etc as is common in the art. As will be understood in the art, the formulation may include an adjuvant to improve the immunogenic effect of the toxoid.

Methods for the production of such toxoid are thus an important aspect of the invention. Toxoids of the present invention may be prepared by simply mixing a solution of a toxin with a solution of a dicarbonyl compound of general structure R—C(O)C(O)R′ (Structure A) until the toxin is substantially inactivated. Suitably, the dicarbonyl compound is added, in solution, to a solution of the toxin and the resulting mixture is incubated at a suitable temperature until the toxin is substantially or completely inactivated. Assays for assessing the activity of a toxin are well known in the art and, for the purposes of the present invention, toxin activity may be assessed by exposing the toxin and, subsequently, the toxoid to a culture of cells that is known to be susceptible to the effects of the toxin. For example, cytotoxicity of toxins to Vero cells may be determined by exposing a culture of Vero cells to a particular concentration of a toxin and monitoring the viability and/or growth of the cells in the culture, to form a baseline measurement. The level of inactivation of the toxin may then be measured by periodically exposing a sample taken from the reaction mixture and exposing to a similar culture of cells and measuring again the viability and/or growth of the cells.

The dicarbonyl compound for use in the method may be any one as described herein, with particularly suitable examples selected from the group consisting of glyoxal, methylglyoxal, butanedione, 1,2-cyclohexanedione and phenylglyoxal. In a particular embodiment, phenylglyoxal is used to produce particularly stable toxoids in a reaction time which is significantly shorter than the time taken for formaldehyde toxoiding. Furthermore the toxoiding reaction may be conducted at room temperature.

Clearly, to achieve substantial or complete inactivation of the toxin, enough dicarbonyl toxoiding reagent must be used to react with the native toxin. For example, with phenylglyoxal, a stoichiometric excess (in relation to the number of freely available arginine residues in the native toxin or “holotoxin”) may conveniently be used be used to ensure that the toxin is substantially or completely inactivated. Large molar excesses of dicarbonyl compound are not necessary and will likely only necessitate that further purification steps are employed. Equimolar amounts of dicarbonyl reagent and toxin may be sufficient and, as exemplified below, very effective and efficient toxoiding is achieved even with low concentrations of toxoiding reagent.

Dicarbonyl reagents such as those described herein have been used previously to modify proteins for a variety of reasons, including: to facilitate binding studies (Herbert) and to elucidate mechanism of action (Belfanz; Watanabe). However, in these examples the molar quantity and concentration of dicarbonyl compound was incredibly high, such that molar ratio of dicarbonyl to toxin ranged from 100:1 to 3100:1. Such ratios are useful for fundamental studies of protein structure and function but are so high as to be unsuitable for pharmaceutical applications. The present invention does not require the use of such high concentrations or molar ratios and is particularly useful for medical applications because low concentrations of toxoiding reagent are utilised.

There is significant advantage in controlling the concentration of the dicarbonyl toxoiding reagent, in that high concentrations of the dicarbonyl compound may distort or change the conformation of the toxin. This is a well-known problem encountered when toxoiding with formaldehyde. The method of the present invention has particular advantages that inactivation may occur at lower concentrations of toxoiding reagent such that the conformational structure of the toxin is maintained after it has been toxoided/inactivated. This is beneficial because the three dimensional structure of the protein is an important factor in ensuring protein integrity and quality. In vaccine and antidote studies it is important to raise antibodies to an antigen that carry as many of the attributes of the original toxin as possible to ensure a high quality antibody response; raising antibodies to unfolded or self-associated proteins carries an unnecessary risk of inducing adverse immunogenic effects and/or may reduce the potency of the antibody serum produced. Maintenance of secondary structure is also an important factor in maintaining stability of a toxoid.

Suitably, therefore, the toxoids of the present invention are prepared using a dicarbonyl compound in solution at a concentration of from about 0.01 mM to about 10 mM, more preferably in the range of from about 0.05 mM to about 5 mM and even more preferably in the range of from about 0.5 mM to about 2.5 mM. A particularly suitable concentration of dicarbonyl compound for use in the method is approximately 1 mM.

In a particular embodiment, phenylglyoxal is used as toxoiding reagent at concentrations of less than or equal to 1 mM to produce a ricin toxoid which is well-folded and which maintains, substantially, the secondary structure of the toxin. The toxoid may be obtained in 24 hours and is highly immunogenic.

The toxoiding reaction is suitably conducted at neutral pH and may be buffered to ensure maintenance of a particular pH throughout the reaction. In a particular embodiment, the dicarbonyl compound in solution is buffered to a pH in the range of from 6 to 14, more preferably between pH7 and 10, and, in a particular embodiment, the reaction is maintained at approximately pH 8.

The toxoid may be prepared at room temperature and pressure. At room temperature and pressure the reaction is still quicker than prior art methods which utilise formaldehyde, producing a completely inactivated toxoid in hours rather than the days or weeks required with formaldehyde. This makes the method particularly suitable for large scale or industrial manufacture of toxoids. In a particular embodiment, however, the reaction is conducted at 37° C. for at least 24 hours. This temperature is commonly used for incubating and culturing biological preparations and is thus already available in the industry and has the advantage that the reaction may complete faster, such that the reaction may be completed overnight.

Suitably, then, the method involves incubation of the reaction mixture at 37° C. for between 8 and 120 hours, and preferably, at 37° C. for approximately 96 hours. These timescales are sufficient to ensure the maximum level of toxin inactivation whilst still being dramatically shorter than the current requirements for formaldehyde toxoiding.

The reaction conditions described above are suitable for producing toxoids from toxins which are derived from a plant, such as abrin or ricin, as described above. Equally, it will be understood that the same method may be used with a toxin which is derived from an animal, such as those hereinbefore described. The method is particularly suitable for producing toxoids of snake toxin. As described above the toxin is suitably derived from a bacterium and, in particular, is selected from the group consisting of botulinum toxin, tetanus toxin, diphtheria toxin, cholera toxin, Bordetella pertussis toxin, pseudomonas endotoxin, shiga toxin or shiga like toxin, anthrax, SEB. It will be well understood in the art, however, that other toxins may be inactivated using the method of the invention

Without being bound by the theory, it is thought that the dicarbonyl toxoiding reagent reacts selectively with guanidine groups on arginine residues, although the level of selectivity is likely to vary throughout the different dicarbonyl groups falling within Structure A. For example, whilst phenylglyoxal is thought to react selectively with guanidine residues to give a product that contains two phenylglyoxal moieties per guanidine group, it is thought that glyoxal, methylglyoxal, butandione and cyclohexanedione are less selective in that they may also react with α- or ε-amino groups in guanidine residues or may also react with other amino acid residues in the toxin. This need not affect the toxoiding capability of the compounds but the relative quantities may be adjusted to account for such side reaction.

The final structure of the toxoid may not need to be fully determined in order for the toxoids to be used as medicaments, vaccines or in the production of antibodies or antitoxins. Consequently, the present invention includes toxoids produced by the method described above and exemplified below. In a preferred embodiment the toxoid is produced using phenylglyoxal. Specific examples of such toxoids include those produced by the phenyl glyoxal inactivation of diphtheria, cholera, clostridium difficile, hemolysin or pseudomonas exotoxin. Of course, the skilled person will understand that these particular toxoids may equally be prepared using substituted phenyl glyoxals such as cyano, fluoro, trifluoromethyl, hydroxy or nitro phenylglyoxal, as well as other compounds falling within general structure A.

The toxoids of the invention are suitably formulated in a pharmaceutical composition as is well known in the art, so that they may be administered directly as prophylactic vaccines or may be used to raise anti-toxoid antibodies, which may then be harvested and used directly as a post-exposure therapy for the treatment of toxin poisoning or intoxication. Alternatively, the antibodies produced may be processed into antibody fragments, may be humanised or otherwise processed to improve their suitability for administration to human subjects.

In a further aspect, therefore, the invention contemplates a pharmaceutical composition comprising the toxoid as described above together with a pharmaceutically acceptable diluent, excipient or carrier. Suitable diluents, excipients and carriers will be known to those skilled in the art. These may include solid or liquid carriers. Suitable liquid carriers include water or saline. The toxoids of the composition may be formulated into an emulsion or alternatively they may be formulated in, or together with, biodegradable microspheres or liposomes.

The composition may further comprise an adjuvant. A further aspect of the invention includes a vaccine composition comprising the toxoid described above, together with a pharmaceutically acceptable diluent or carrier and an adjuvant.

Many adjuvants will be suitable for inclusion in the vaccine composition provided that the adjuvant stimulates an immune response in a host to whom the composition is administered. Particularly suitable adjuvants include, but are not limited to, ISCOMs™ (Quillaja saponins), CpG oligodeoxynucleotides, Alhydrogel, MPL+TDM, Freunds Complete and Freunds Incomplete Adjuvant.

Such compositions and toxoids are suitable for use as vaccine against the toxin from which the toxoid was derived.

Additionally or alternatively, the toxoids described above may be used in the manufacture of an antibody for treatment of intoxication by a toxin.

Consequently, an antibody produced by the toxoid forms a further aspect of the invention. Such antibodies are particularly useful in the treatment of toxin poisoning or intoxication.

In yet a further aspect the invention provides a method of producing an antitoxin which comprises administering to an animal a toxoid as described above in an amount effective so as to induce anti-toxoid antibodies and taking blood from said animal, separating serum from the blood and extracting antibodies from the serum. The extracted antibodies may then be purified using methods as are known in the art.

The polyclonal antibodies may be generated by immunisation of any animal (such as rabbit, rat, chicken, goat, horse, sheep, cow etc) routinely used for generation of therapeutic antibodies. The immunization of the animal may utilize an adjuvant as is necessary. Conveniently the polyclonal antibodies may be derived from the same or from several batches of antisera, which may be combined.

The antibodies so produced may be used directly or formulated into pharmaceutical compositions as is known in the art. The antibody may be humanized using conventional methods, or comprise a chimeric antibody. Alternatively the antibodies are fragmented to produce despeciated antitoxin antibody fragments.

Fragments of the antibodies may be large in that they comprise a significant proportion of the antibody from which they are derived. For instance, a large fragment will comprise the entire variable domain, as well as some of a constant region (Fc). In particular, large antibody fragments include F(ab′)₂ or F(ab)₂ fragments but they may also comprise deletion mutants of an antibody sequence. In particular the large binding fragment is F(ab′)₂.

Such large binding fragments are also suitably derived from polyclonal or monoclonal antibodies using conventional methods such as enzymatic digestion with enzymes such as pepsin to produce F(ab′)₂ fragments. Alternatively the fragments may be generated using conventional recombinant DNA technology, provided that the antibody sequence is determined.

Small fragments of antibodies may also be used. Such small fragments will include antibody fragments which lack a significant element of the antibody from which they are derived, for example, it may lack a significant portion of the Fc chain, provided it retains the ability to bind to the toxin. In particular small fragments of antibodies include Fab and Fab′ fragments as well as single chain (sc) antibodies, FV, VH and VK fragments.

An advantage of antibody fragments is that they are able to reduce the risk of unwanted side-effects when administering antibodies which have been derived from an animal source.

Additionally, combinations of antibodies with fragments of antibodies are envisaged within the scope of the invention. Such compositions comprising mixtures of whole antibodies with large or small fragments or of large and small fragments are beneficial as they may provide rapid and sustained antitoxin activity.

One factor that affects the window of opportunity in the treatment of toxin intoxication is the speed with which the antitoxin is distributed around the body to the sites of action of the toxin. Whole antibodies and large antibody fragments are, due to their size, likely to be less extensively distributed into the extravascular space than small antibody fragments. Small fragments, on the other hand, are likely to provide an antitoxin capability that penetrates rapidly into the extravascular space to give rapid protection.

The binding of antibodies and fragments to toxins is normally reversible and therefore there is a risk of a “rebound” effect due to toxin being released, unless at least some functional antibody or antibody fragment remains in the plasma to bind any released toxin. Rapid clearance of small antibody fragments means that they are less available to “mop up” and neutralise any released toxin. However, by virtue of their greater size and slower clearance rate, whole antibodies and large fragments will be more widely available in the plasma and, consequently, are able to neutralise any released toxin, thereby minimising the rebound effect. The slower clearance rate and longer persistence in the plasma means that whole antibody and large fragments of antibodies provide prolonged protection. Consequently, the “prime-boost” effect of such compositions makes them a particularly preferred embodiment of the invention.

The toxoids and the anti-toxoid antibodies or fragments produced (i.e. anti-toxins) are particularly useful in the treatment of toxin intoxication

Consequently, further aspects of the invention are formed by a method of vaccinating against intoxication by a toxin, comprising administering to a mammal a pharmaceutically effective amount of a toxoid as described above or of a pharmaceutical composition or of a vaccine composition as described herein and by a method of treating an intoxicated mammal, including man, comprising administering to the mammal a pharmaceutically effective amount of the anti-toxoid antibodies (and/or compositions of antibodies and fragments or large and small binding fragments) as described above.

The invention will now be described by way of example, with reference to the following drawings in which:

FIG. 1 shows the general chemical structure of the dicarbonyl toxoiding reagent of the present invention (structure A) and the chemical structure of one such reagent, phenylglyoxal, which corresponds to general structure R—C(O)C(O)H.

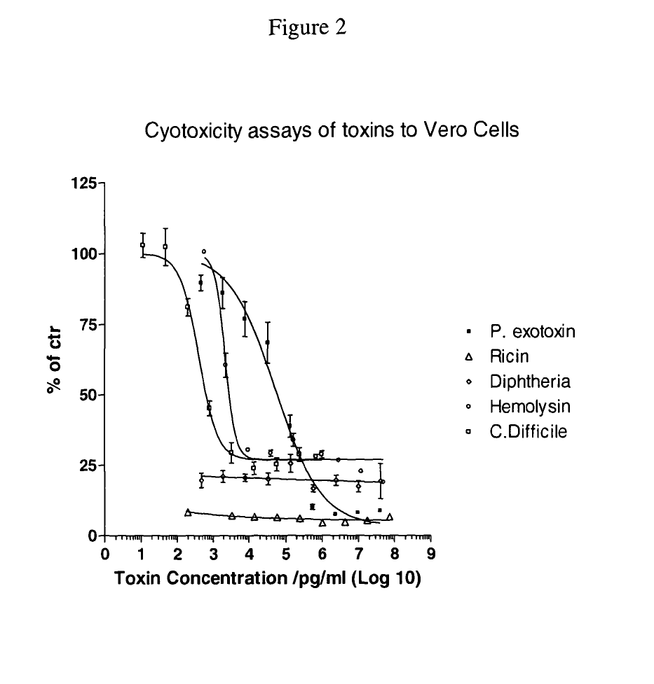

FIG. 2 shows the cytotoxicity of various toxins to Vero cells (The Vero cell line is derived from kidney epithelial cells of the African Green Monkey.)

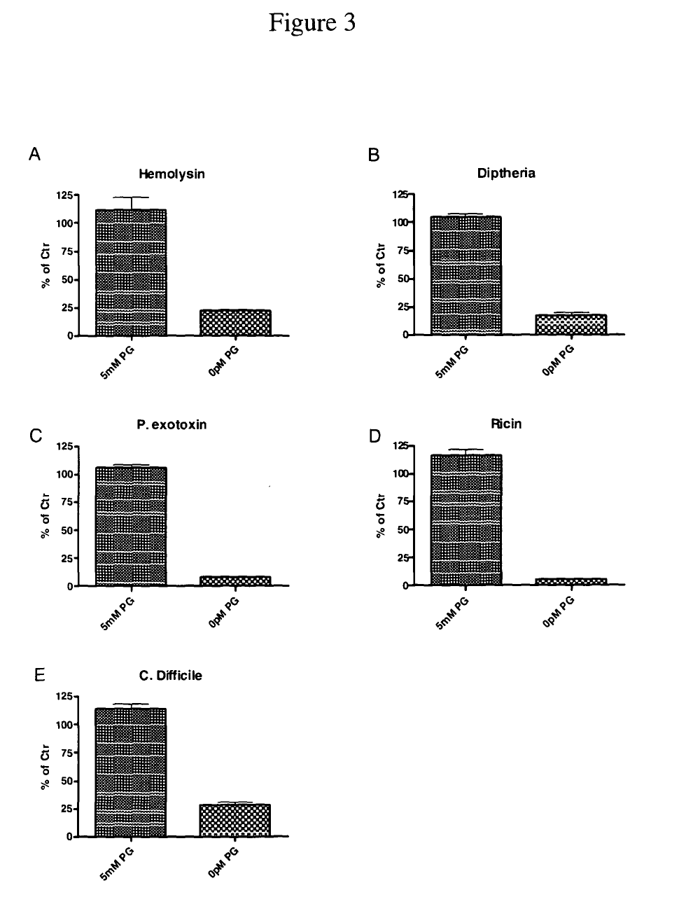

FIG. 3 shows the effect of toxoiding (inactivating) toxins using the α-dicarbonyl toxoiding agent phenylglyoxal (5 mM) for 7 days at 37° C. FIG. 3A=Hemolysin, FIG. 3B=Diphtheria, FIG. 3C=pseudomonas exotoxin, FIG. 3D=ricin, FIG. 3E=Clostridium difficle toxin

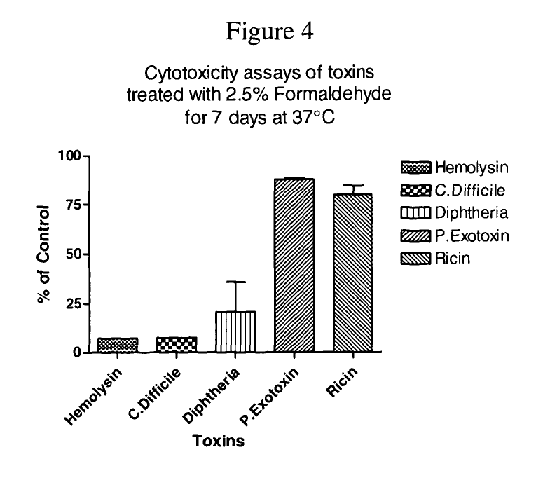

FIG. 4 shows the effects of 2.5% (m/v) formaldehyde inactivation under the same conditions (7 days at 37° C.) as described for PG in FIG. 3. The equivalent molar concentration of formaldehyde is 0.625M.

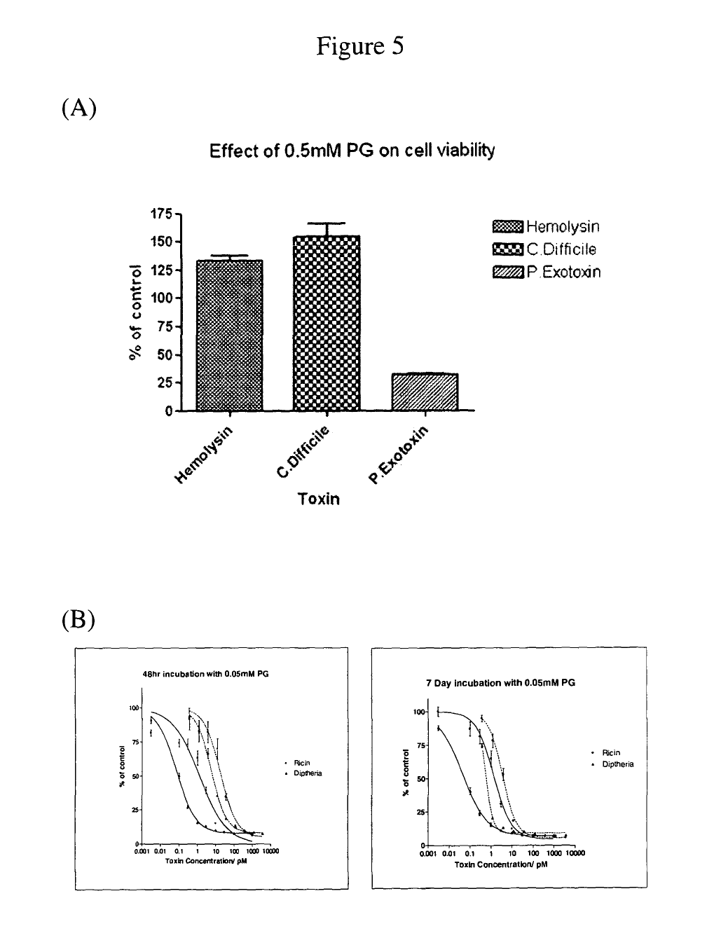

FIG. 5 shows the effect of (A) 0.5 mM phenylglyoxal on various toxins; higher cell viability demonstrating that the toxin has been inactivated and (B) cytotoxicity of ricin and diphtheria toxoided with 0.05 mM PG over 48 hours and 7 day time periods. (Solid lines are toxin alone and hashed lines show the toxin+PG)

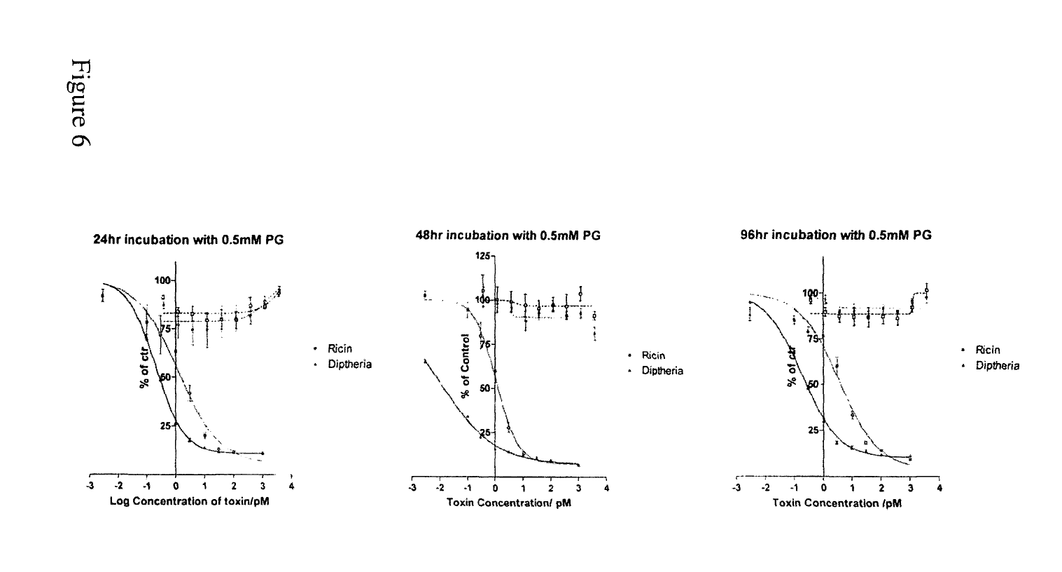

FIG. 6 shows the effect of time on incubation of toxin with 0.5 mM phenylglyoxal over 24, 48 and 96 hours.

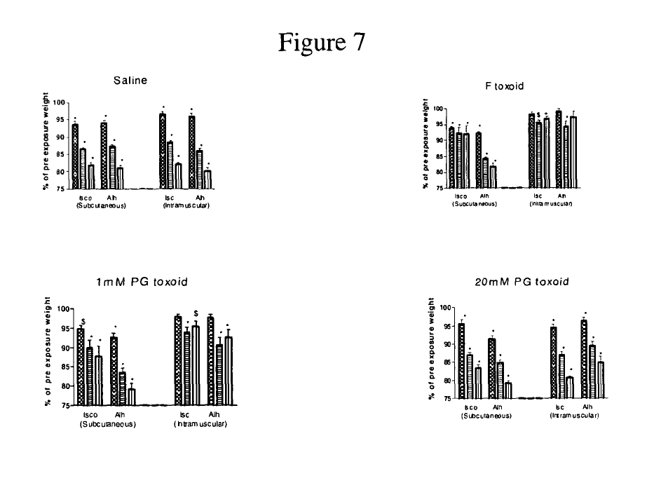

FIG. 7 shows a comparison of body weight between the vaccine groups over the first three days following ricin challenge (PG versus formaldehyde)

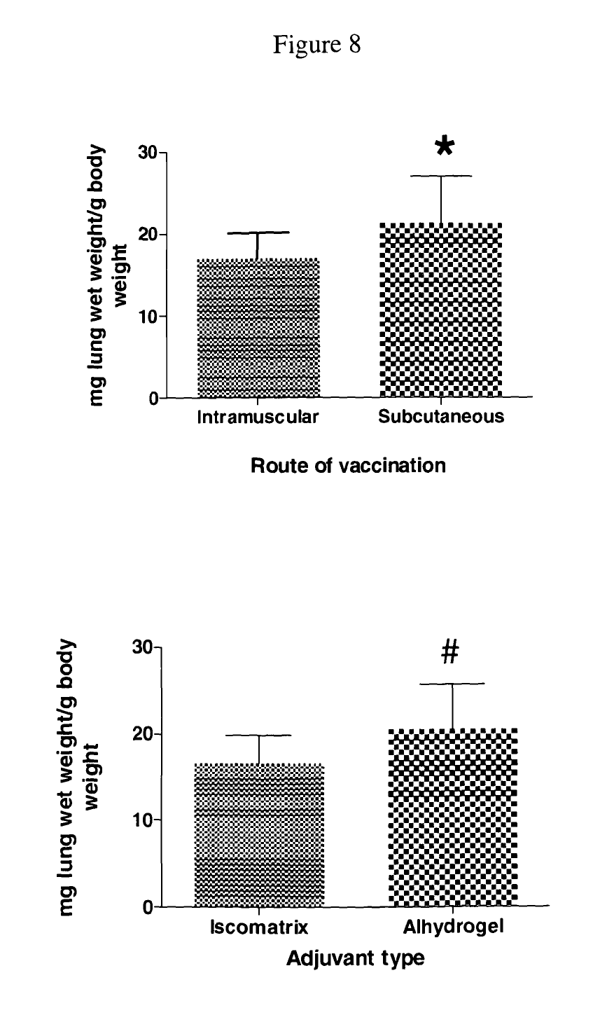

FIG. 8 shows wet lung weight data from all surviving animals culled at day 7 post ricin challenge and compares the effect of route, toxoid and adjuvant for 1 mM PG and formaldehyde (F) toxoid groups.

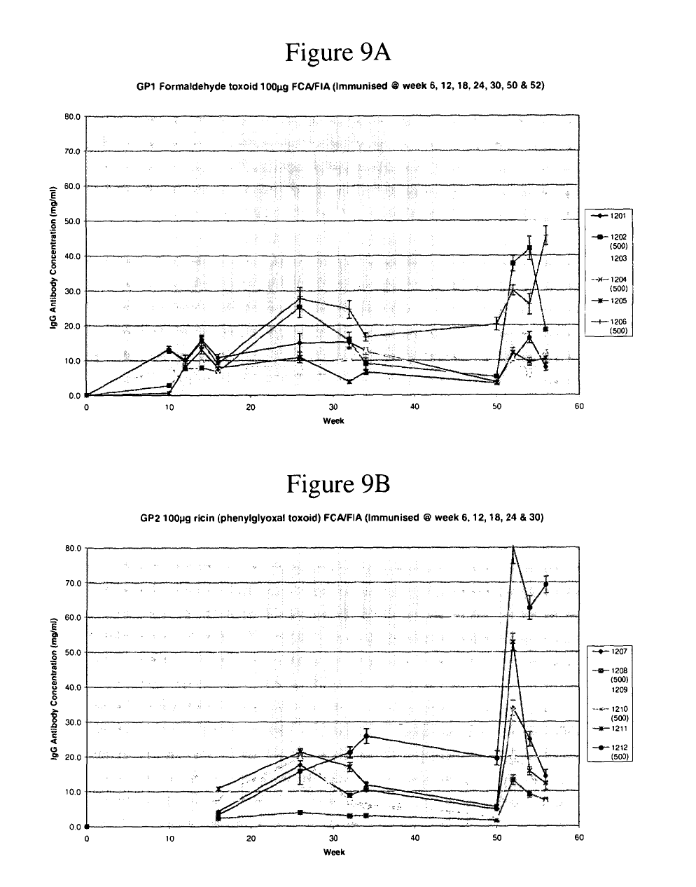

FIG. 9 shows ELISA antibody titres from sheep vaccinated with ricin toxoid prepared with formaldehyde (FIG. 9A) and phenylglyoxal (FIG. 9B)

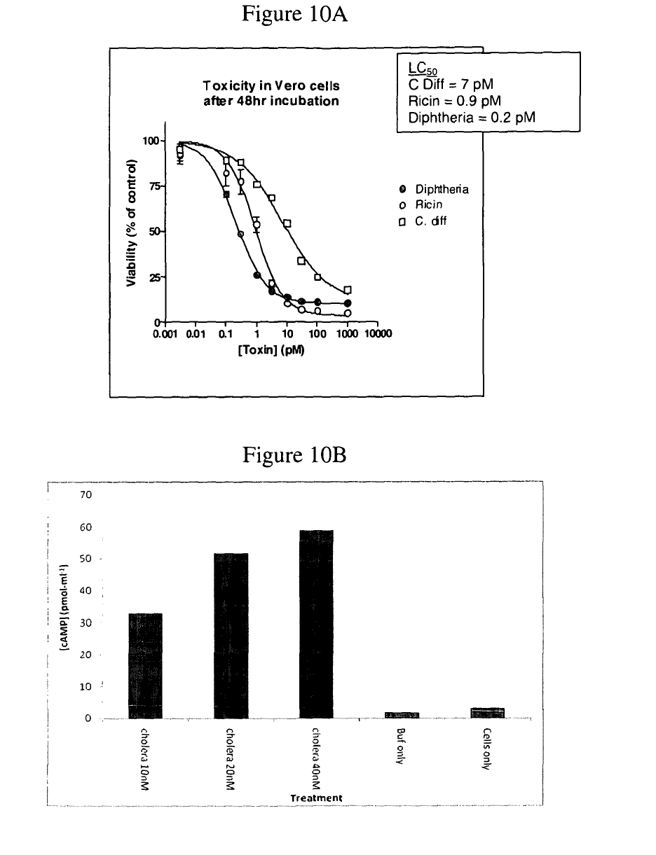

FIG. 10 shows cytotoxicity of toxins on Vero cells (Diphtheria, ricin and C. difficile) (FIG. 10A) and the amount of cAMP released into supernatant of Vero cells after a 1 hr incubation with increasing concentrations of cholera toxin (comparitive assay for assessment of the toxicity of cholera toxin)

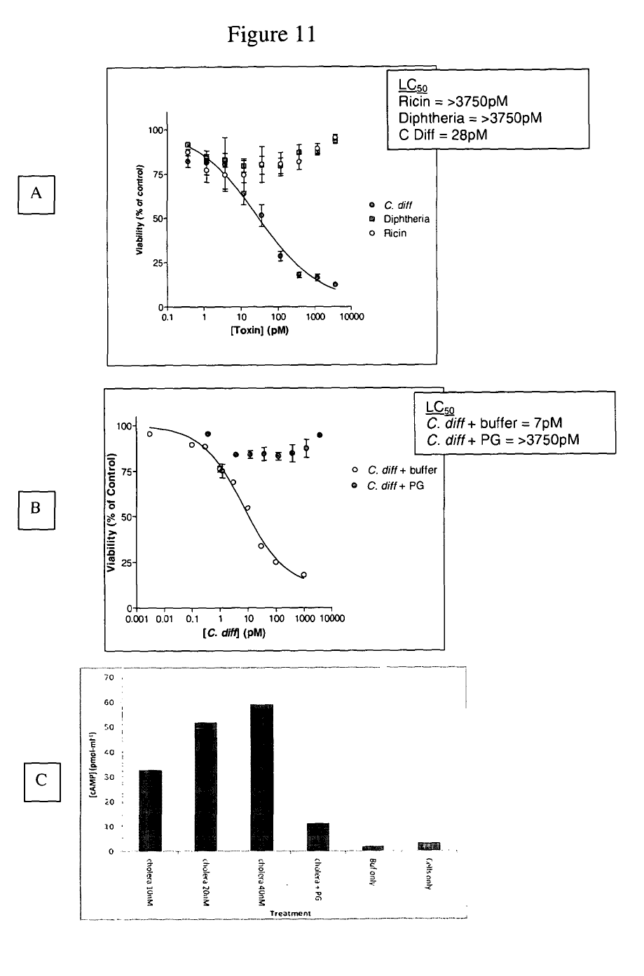

FIG. 11 shows cytotoxicity of Vero cells exposed to toxins treated for 24 hrs with 0.5 mM PG (A), cytotoxicity of Vero cells exposed to C. diff toxin treated for 7 days with 0.5 mM PG (B), and concentration of cAMP released from Vero cells after a 1 hr exposure to 40 nM of cholera toxin toxoided with 0.5 mM PG for 7 days compared to control cells.

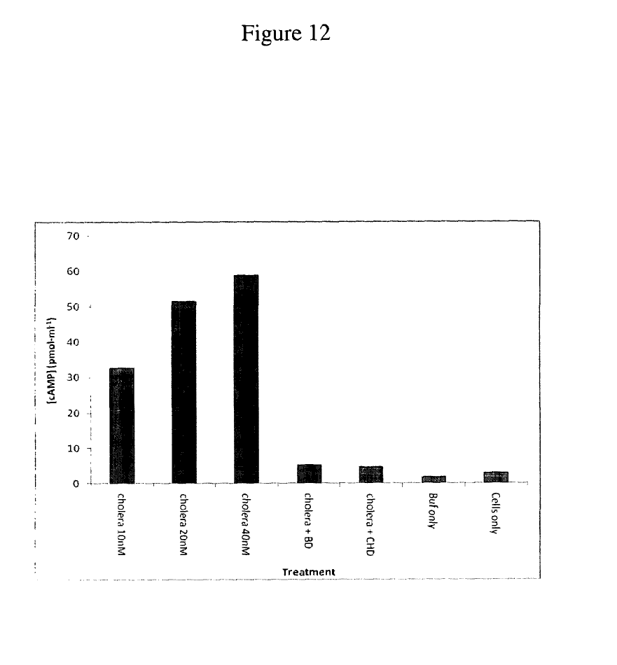

FIG. 12 shows concentration of cAMP released from Vero cells after a 1 hr exposure to 40 nM cholera toxin toxoided with either 0.5 mM CHD or 5 mM BD for 7 days compared to control cells.

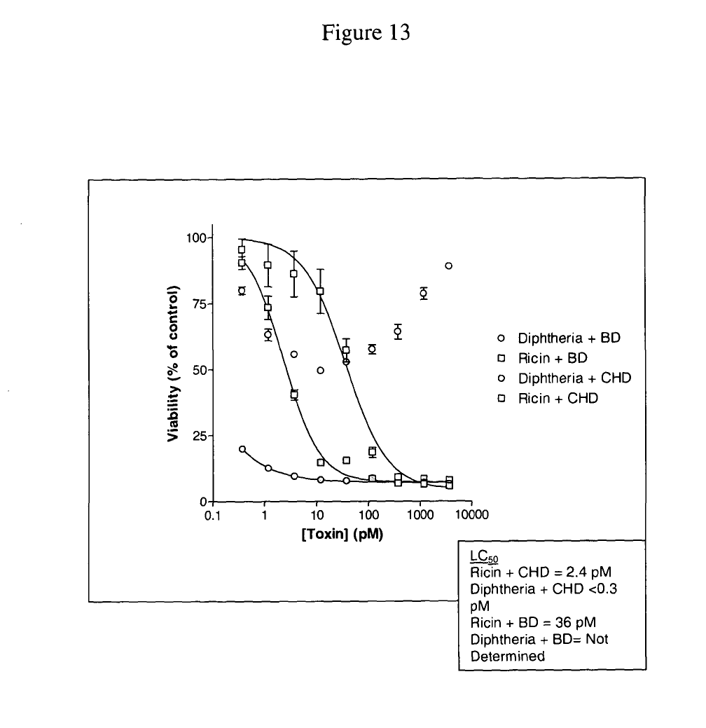

FIG. 13 shows cytotoxicity of Vero cells exposed to toxins treated for 7 days with 0.5 mM BD or CHD. An accurate LC₅₀ for BD-treated Diptheria toxin could not be determined.

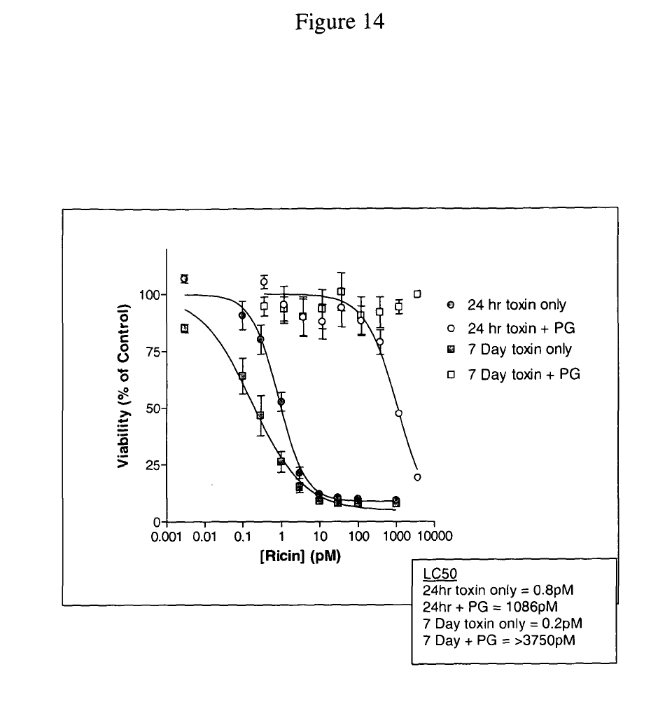

FIG. 14 shows cytotoxicity of Vero cells exposed to ricin treated with 0.5 mM PG at room temperature for either 24 hours or 7 days

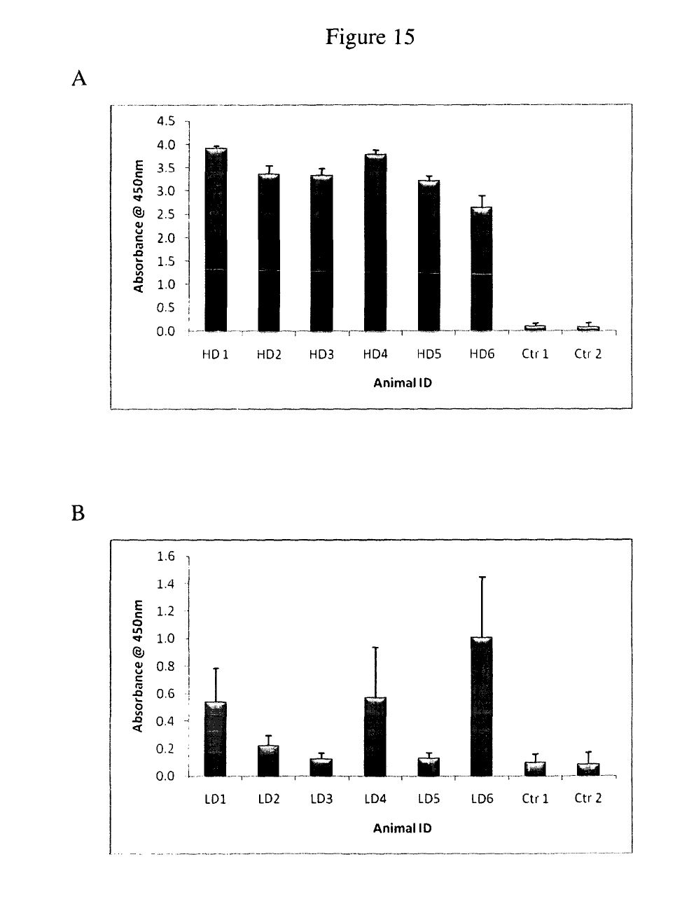

FIG. 15 shows serum antibody levels of animals immunised with high dose toxoid (2.5 μg/mouse) shown in graph A and low dose toxoid (0.625 μg/mouse) shown in graph B displayed by absorbance at 450 nm. (HD, high dose; LD, low dose; Ctr, naïve control)

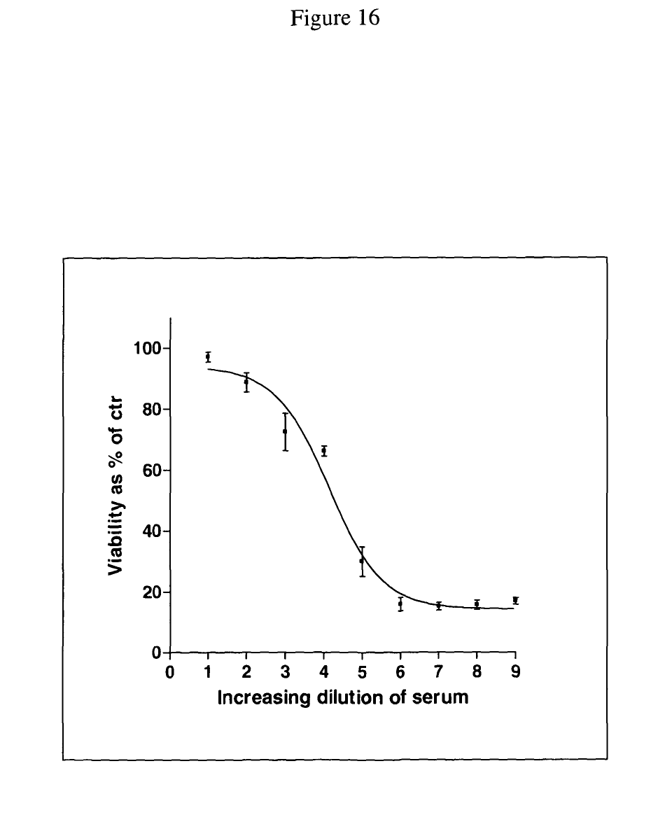

FIG. 16 shows the results of a neutralising assay with Vero cells exposed to increasing concentrations of immunised animal serum incubated for 1 hr with C. difficile toxin A

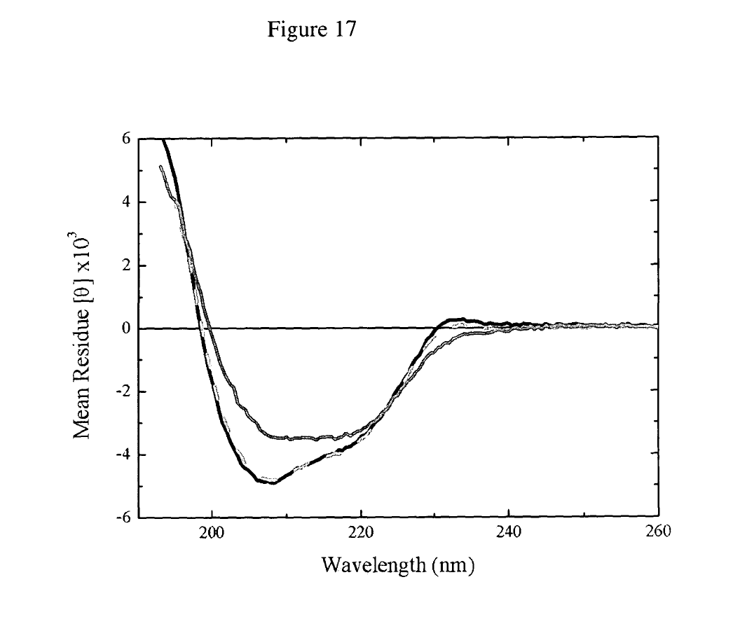

FIG. 17 shows far-UV CD spectra in 10 mM phosphate buffer pH7 of ( [symbol] ) whole native ricin (0.186 mg/ml), (---) 1 mM PG ricin toxoid (24 hr, 37° C.), ( [symbol] ) 300 mM formylated ricin toxoid.

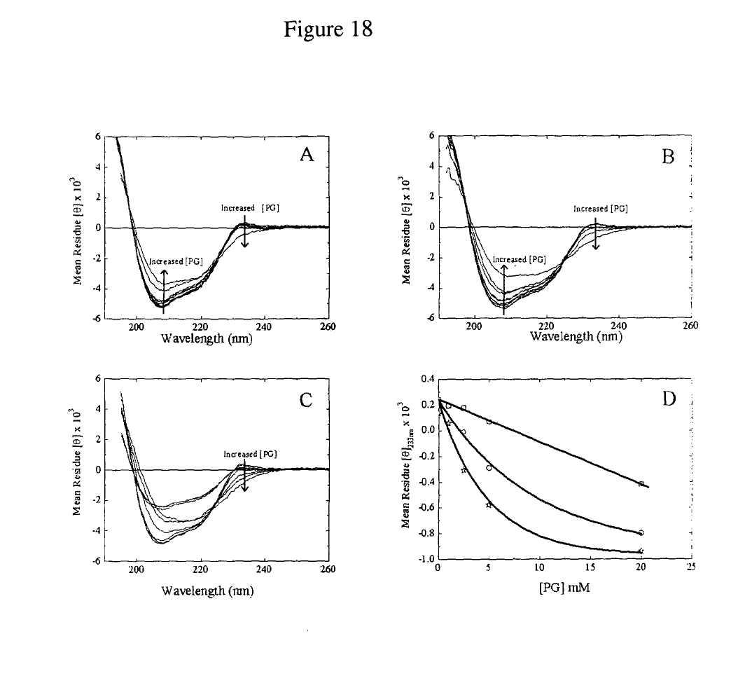

FIG. 18 shows the far-UV CD spectra in 10 mM phosphate buffer (pH7) of whole native ricin and ricin PG toxoids as a function of PG concentrations (0, 0.001, 0.01, 0.05, 0.1, 0.5, 1.0, 2.5, 5.0 & 20.0 mM) incubated at: (A) RTP for 24 hr, (B) RTP for 96 hr and (C) 37° C. for 24 hr. (D) Plot of mean residue molar elliptiticity at 233 nm versus PG concentration: -□- (24 hr, RTP), -∘- (96 hr, RTP) and -★- (24 hr, 37° C.).

### EXAMPLE 1

Cytotoxicity of Toxins to Vero Cells (Cell Viability Assay)

Toxins were purchased from Sigma-Aldrich, Dorset, UK except for ricin (Zan 030) which was prepared at the Defence Science and Technology Laboratory, from seeds of Ricinus communis var. zanzibariensis according to the method of Griffiths et al 1995 (Griffiths, G. D., Rice, P., Allenby, A. C., Bailey, S. C., and Upshall, D. G. (1995). Inhalation Toxicology and Histopathology of Ricin and Abrin Toxins. Inhalation Toxicology 7(2), 269-288), and were used in the following quantities:
Hemolysin (1 mg)
Clostridium difficile (2 μg)
Diphtheria (1 mg)
Pseudomonas exotoxin (0.5 mg)

All toxins were diluted with sterile PBS to give a final concentration of;

| | |
|---|---|
| Hemolysin | 1.0 mg/ml |
| Clostridium difficle | 4.0 μg/ml |
| Dipthteria | 1.0 mg/ml |
| Pseudomonas exotoxin | 1.0 mg/ml |
| Ricin | 1.6 mg/ml |

The toxin solutions were prepared in 50 microlitre aliquots and were frozen at −80° C., until required for use.

Cytotoxicity of toxins to Vero cells was initially determined as shown in FIG. 2. It is shown clearly in FIG. 2 that each of the toxins was toxic to Vero cells, as demonstrated by the very low levels of cell viability achieved after 48 hrs exposure to increasing concentrations of toxin. It is noteworthy that ricin and diphtheria were toxic to Vero cells at all concentrations tested, demonstrating that the limit of toxicity for these toxins is very low.

### EXAMPLE 2

Effect of Toxoiding on Toxin Activity

The ability of dicarbonyl toxoiding reagents to inactivate (toxoid) the toxins described in Example 1 was demonstrated using phenylglyoxal (“PG”) (FIG. 3A) at 5 mM concentration, made up in bicarbonate buffer pH 8.0. As a comparison, inactivation with formaldehyde (2.5%) was also performed to compare the toxoiding effects of PG with the commonly used formaldehyde. The toxin solutions were incubated at 37° C. for seven days to ensure complete inactivation with formaldehyde was achieved.

The results are presented in FIG. 3. As is evident from the figures, exposure to 5 mM PG for 7 days at 37° C. completely inactivated all toxins. The control data set refers to untreated toxins at the equivalent concentration assessed for the toxoids.

As shown in FIG. 4, even after 7 days, not all of the toxins had been inactivated by formaldehyde treatment.

### EXAMPLE 3

Effect of Concentration of Toxoiding Ability

The experiment described in example 2 was repeated using lower concentration of PG i.e. 0.5 and 0.05 mM. At a concentration of 0.5 mM, three out of the five toxins were completely inactivated (hemolysin, C. difficile and cholera). This is shown in FIG. 5A. No toxoiding effect was observed with 0.05 mM PG (as shown in FIG. 5B). This demonstrates, at least, that, at concentrations above 0.5 mM, PG is a potent toxoiding agent.

### EXAMPLE 4

Rate of Toxin Inactivation with Phenylglyoxal

The length of time taken for complete inactivation of toxins, ricin and diphtheria, was assessed using the assays and methods described above, but with incubation of the toxin and PG being terminated after 24, 28 and 96 hours. The results are presented in FIG. 6 and clearly demonstrate that, even after only 24 hours, 0.5 mM PG is able to completely inactivate both ricin and diphtheria toxins. This suggests that PG has significant advantages over the traditional formaldehyde treatment in that complete inactivation was achieved with a low concentration of toxoiding reagent in a remarkably short period of time.

### EXAMPLE 5

Inactivation of C. difficile Toxin

The experiment described in Example 4 was conducted for C. difficile but inactivation was not complete after 48 hours. However, complete inactivation was observed after 7 days, which still represents a vast improvement over the time traditionally taken for formaldehyde treatment (3 weeks). These results are shown in Table 1

TABLE 1
Cell viability on exposure to toxin
before and after inactivation with PG

| | | |
|---|---|---|
| Toxin | LC50/pM | LC50 + PG/pM |

24 hr incubation with 0.5 mM PG

| | | |
|---|---|---|
| Ricin | 1.2 | >3750 |
| Diptheria | 0.2 | >3750 |
| C Difficile | 4.4 | 27.7 |

48 hr incubation with 0.5 mM PG

| | | |
|---|---|---|
| Ricin | 1.1 | >3750 |
| Diptheria | 0.2 | >3750 |
| C Difficile | 4.8 | 44.7 |

96 hr incubation with 0.5 mM PG

| | | |
|---|---|---|
| Ricin | 3.6 | >3750 |
| Diptheria | 0.2 | >3750 |
| C Difficile | n/a | n/a |

7 Day incubation with 0.5 mM PG

| | | |
|---|---|---|
| Ricin | n/a | n/a |
| Diptheria | n/a | n/a |
| C Difficile | 20.8 | >3750 |

### EXAMPLE 6

Quality of Protection Achieved Using Alternative Ricin Toxoids and Adjuvant Combinations

Ricin toxoids were prepared as described above and the ability of the toxoid to elicit a protective immune response was demonstrated by comparing the immunogenicity and the protective efficacy of a novel ricin vaccine candidate (phenyl glyoxal (PG) holotoxin toxoid) against a standard ricin vaccine formulation (formaldehyde inactivated ricin holotoxin) following exposure of Balb/C mice to 5LCt50 of aerosolised ricin. Vaccine candidates were administered either by the subcutaneous or intramuscular routes and formulated with either alhydrogel (20% v/v) or Iscomatrix (5 μg/animal). Humoral antibody (IgG1 and IgG2a) responses to vaccine antigens were measured prior to ricin challenge. The quality of protection was also monitored by measuring, survival, body weight change and the signs and symptoms of ricin intoxication for up to 7 days post toxin challenge. 20 mM PG toxoid failed to elicit a protective immune response, regardless of route of administration or adjuvant type. This toxoid was as ineffective as the saline controls. Immunisation with 1 mM PG toxoid induced a strong Th2 response which was characterised by elevated concentrations of ricin specific IgG1 in the blood, giving a quality of protection following exposure which was similar to that found with formaldehyde toxoid (F toxoid). Subcutaneous immunisation and formulations with alhydrogel, of both F and PG toxoids, were found to offer lower protective quality than the intramuscular route and Iscomatrix. This experiment indicates that PG toxoided ricin holotoxin formulated with Iscomatrix is atleast as good as the current F toxoid formulated with alhydrogel and may even be superior in certain properties.

Ricin Purification

Crude ricin was prepared in house from seeds of Ricinus communis var. zanzibariensis according to Griffiths et al 1995 (supra). Briefly, seeds were homogenised in sodium chloride and then acidified to pH 4.8 with HCl prior to overnight stirring at 4° C. The next day the homogenate was centrifuged to remove seed debris and the supernatant clarified with petroleum ether. The resultant soluble proteins were then precipitated by centrifugation (300 g) following addition of excess ammonium sulphate and the precipitate recovered. The precipitate was re-suspended in phosphate buffered saline (PBS), filtered, dialysed to remove any remaining ammonium sulphate and then stored in 20 ml aliquots prior to use. On the day of use, the crude toxin solution was centrifuged at 13,400 g for 5 minutes to remove newly formed solid debris and the resultant solution prepared for inhalation studies. Pure ricin holotoxin for enzyme linked immunosorbant assay (ELISA) studies was prepared according to the full method described in (Griffiths et al. 1995, supra).

Toxoids

F toxoid was prepared following the addition of formaldehyde (37% v/v) to ricin (1 mg/ml) to give a final concentration of 2.5% v/v (equivalent to 0.625M formaldehyde). This mixture was incubated at 37° C. for 3 weeks after which lysine was added to a final concentration of 0.1M. The solution was then desalted into saline using PD10 chromatography columns. PG toxoid was prepared by the addition of equal volumes of toxin (2 mg/ml) and phenyl glyoxal (2 or 40 mM in bicarbonate buffer, pH 8) to give a final concentration of 1 mg/ml toxin in 1 and 20 mM phenyl glyoxal. The solutions were then incubated at room temperature and pressure for 96 hours. After incubation, the PG toxoid was desalted into saline using PD10 chromatography columns. Protein concentrations of each toxoid were measured using a previously published method (Griffiths et al. Vaccine 17 (1999) 2562-2668). Aliquots of both toxoids were stored frozen at −80 degree C. until use.

Animal Groups

Female Balb/C mice (Charles-River Laboratories, Margate, Kent, UK: 20 g bodyweight) were divided into four vaccine groups (n=24 animals/group) consisting of saline, F toxoid, 1 mM PG toxoid and 20 mM PG toxoid. Animals from all of these groups were further subdivided into groups formulated with either Alhydrogel® (20% v/v) or Iscomatrix™ (5 ug/animal) and administered via one of two routes; subcutaneous (s.c.) or intramuscular (i.m) to give a total of 16 groups (n=6 animals/group). Mice were injected with vaccine formulation (5 μg/kg body weight) on days 0 and 21 and on day 35 all animals were exposed to 5LCt50 of aerosolised crude ricin. Twenty four hours prior to toxin challenge, a tail vein blood sample (100 microlitres) was taken, incubated at 4 degree C. overnight and then centrifuged at 13,400 g for 1 min. The separated serum was removed and stored at −20 degree C. prior to the measurement of ricin specific IgG1 and IgG2a concentrations.

Ricin Specific Serum Antibody Measurements

Antibody levels for ricin specific serum IgG1 and IgG2a were determined using an indirect ELISA, with samples analysed against a standard curve of purified murine immunoglobulin (IgG1 or IgG2a). Row A of a ninety-six well microtitre plate (Immulon HB polystyrene flat bottomed microtitre plate—Thermo Scientific, Basingstoke, UK) was coated with goat anti-mouse IgG1 or IgG2a at 5 μg/ml (100 μL per well) (AbD Serotec, Oxford, UK) in PBS. Remaining wells were coated with purified ricin holotoxin at 5 μg/ml (100 μL per well) in PBS and the plates incubated at 40 C overnight. The following day the wells were washed three times with 300 μl per well of wash buffer (0.05% Tween in 1×PBS) using an automated plate washer (Thermo Labsystems Ultrawash Plus). The wells were then blocked by the addition of 100 μL of 1% (w/v) Blotto (non-fat dry milk powder—Biorad, Hemel Hempstead, UK) in PBS, at 37° C. for 1 hour. Following blocking a standard curve of purified mouse IgG1 or IgG2a (AbD Serotec, Oxford, UK) was created using serial dilutions in Row A with a starting concentration of 10 μg/ml. Serum samples (diluent: 1% (w/v) Blotto in PBS) were analysed in duplicate and serially diluted across the plate (Rows C-H). A previously identified standard quality control serum sample (pooled murine sera post ricin exposure) was also analysed to confirm inter-plate variability (Row B), along with a diluent only blank. Plates were incubated for 1 hour at 37° C. and then washed (as above). Ricin specific IgG1 and IgG2a were detected by goat anti-mouse IgG1:HRP (1 in 2000 dilution in diluent) and IgG2a:HRP (1 in 1000 dilution in diluent) (AbD Serotec, Oxford, UK) for 1 hour at 37° C. A final wash step was carried out prior to the addition of the colorimetric substrate consisting of ABTS (2,2′-azino-bis(3-ethybenzthiazoline-6-sulphonic) acid) and H2O2 (0.01% v/v) in citrate phosphate buffer. Plates were incubated at 37′C. for 30 minutes allowing the colour to develop. Plates were read at 414 nm (Thermo Labsystems Multiskan Ascent plate reader) and analysed.

Sample antibody levels were determined by comparison with the standard curve and expressed as relative micro g/ml IgG1 and IgG2a. The term relative refers to the understanding that there may be a difference in the binding kinetics between the anti species immunoglobulin and ricin specific immunoglobulin binding to their respective targets. Samples were analysed in the Thermo Labsystems Multiskan Ascent plate reader using a four-parameter logistic (PL) curve (formula: y=b+(a−b)/(1+xc)d; where a=maximum signal, b=minimum signal, c=concentration at inflection point, d=slope). Sample IgG1 and IgG2a levels were read off the linear part of the 4PL curve and where parallelism was optimal. Inter-plate variability, as determined by the quality control data, was less than 30% CV.

Inhalation Challenge

Ricin aerosol was generated from a solution of crude ricin in PBS (containing 20 μg/ml fluorescein) using a Liu and Lee constant output nebuliser and generating an aerosol concentration of 6.36 μg protein/litre of air (5×LCt50). The LCt50 was defined by previous in-house studies. The animal exposure system consisted of a horizontal 1.4 metre long glass tube with 12 ports, 6 along each side. The animals were loaded into plethysmography tubes (for real time respiratory monitoring) and attached to the ports. The heads of the animals projected into the aerosol stream and toxic material was prevented from reaching the body by a latex diaphragm fitted snugly around the neck. To estimate the aerosol concentration, aerosol was trapped on a full-flow in-line glass fibre filter, which was recovered after each run. The filter was disintegrated by mechanical shaking in 50 ml of PBS. An aliquot of the filter suspension was taken, centrifuged at 13,400 g for 5 minutes and an appropriate dilution of the supernatant was measured fluorometrically (513 nm excitation and 487 nm emission). The concentration of toxin in the aerosol was calculated on the basis of the quantity of captured fluorescein, the known ratio of fluorescein to toxin in the original sample, the exposure time (10 minutes) and the total volume of air that had passed over the noses of the animals. The inhaled dose for each animal was calculated from the inhaled volume (from real time plethysmography data) and the aerosol concentration.

Animals were removed from the inhalation line after exposure and returned to their home cages. Survival, bodyweight and signs and symptoms of ricin intoxication were recorded every day for 7 days. All surviving animals on day 7 were killed by an overdose of Euthatal (0.7 ml/kg) and the lungs removed and weighed.

Statistical Analysis

One way analysis of variance was undertaken to establish an effect of route, adjuvant and toxoid on minute volume. Three way analysis of variance, was undertaken on circulating ricin specific antibody concentrations (IgG1 and IgG2a) and body weight (with repeated measures for time), also to establish an effect of route, adjuvant and toxoid. Significances between groups were further identified using the Bonferroni post hoc-test.

The Fischer Exact test and ordinal logistic regression analysis was undertaken to establish an effect of route, adjuvant and toxoid on survival and signs and symptoms of ricin intoxication, respectively. Correlations between survival, body weight changes and IgG1 and IgG2a concentrations were undertaken using the Pearson rank test.

In all cases a p value less than 0.05 was considered significant.

Results

Antibody Response

Circulating ricin-specific IgG1 and IgG2a levels obtained pre-exposure to inhaled ricin are outlined in Table 2. Regardless of route of toxoid administration or adjuvant type, 20 mM PG toxoid failed to initiate a consistent or robust immune response and which was not statistically significant then that obtained for the saline controls.

F and 1 mM PG toxoids induced strong ricin specific IgG1 responses with concentrations being generally higher in those animals administered F toxoid. On further investigation a complex interaction of route, toxoid and adjuvant was observed. Generally, administration of toxoids by the intramuscular route produced higher antibody responses. The presence of Iscomatrix also produced higher antibody levels than alhydrogel.

Formulation of both toxoids with Iscomatrix, by either route, elicited a mild Th1 response as indicated by increased circulating levels of IgG2a.

Inhalation Data

To ensure that observed differences in survival were not due to increased levels of inhaled toxin, all groups were compared with respect to minute volume, and hence total inhaled toxin. No statistically significant differences were observed between any of the groups during ricin aerosol challenge (range: 47.8 ml.min-1-67.5 mls.min-1, p=0.77).

TABLE 2
The effect of route, adjuvant and toxoid on circulating ricin specific IgG₁ and IgG₂ₐ
concentrations 24 hours prior to exposure to 5 × LCt₅₀ of aerosolised crude ricin

| | Alhydrogel | | | | Iscomatrix | | | |
|---|---|---|---|---|---|---|---|---|
| | Subcutaneous | | Intramuscular | | Subcutaneous | | Intramuscular | |
| | IgG₁ | IgG₂ₐ | IgG₁ | IgG₂ₐ | IgG₁ | IgG₂ₐ | IgG₁ | IgG₂ₐ |
| | (μg/ml) | (μg/ml) | ( μg/ml) | (μg/ml) | (μg/ml) | (μg/ml) | (μg/ml) | (μg/ml) |
| Saline | ND | ND | 0.2 ± | ND | ND | ND | ND | ND |
| | | | 0.4 | | | | | |
| 20 mM PG | 0.7 ± | ND | 0.0 ± | ND | ND | ND | ND | ND |
| | 1.4 | | 0.0 | | | | | |
| 1 mM PG | 4.4 ± | ND | 13.8 ± | ND | 62.4 ± | 3.4 ± | 31.4 ± | 0.4 ± |
| | 5.0 | | 8.6 | | 64.2# | 0.4 | 36.3+ | 0.7 |
| Formaldehyde | 9.2 ± | ND | 43.0 ± | ND | 46.8 ± | 1.7 ± | 138.9 ± | 1.7 ± |
| | 8.6+ | | 32.4* | | 44.3+ | 0.5 | 35.7* | 0.5 |

Survival

All animals were monitored daily for 7 days following exposure to 5×LCt50 of aerosolised crude ricin. Deaths were recorded and shown in Table 4. All control (saline with adjuvant) animals died between days 3 and 5. This was not significantly different to that seen for the 20 mM PG toxoid group.

Animals vaccinated with either F or 1 mM PG toxoid had significantly higher rates of survival after ricin challenge than control animals, but were not significantly different from each other. However, on closer scrutiny of the individual groups, only 50% of those animals immunised via the subcutaneous route with 1 mM PG toxoid formulated with alhydrogel survived. When survival was compared with circulating ricin specific IgG1 concentrations (data not shown), a significant positive correlation (p<0.05) was observed indicating that those animals which died tended to have the lowest concentrations of protective antibodies.

TABLE 3
A comparison of circulating concentrations of ricin specific IgG₁ with survival, clinical score and
percentage body weight in mice vaccinated with 1 mM PG and F toxoid

| | | | | | |
|---|---|---|---|---|---|
| | IgG1 | | | | % of body |
| Animal | (relative | Survival | Survival | Clinical | weight at |
| No. | μg/ml) | at day 3 | at day 7 | Score | day 3 |

A—1 mM PG Toxoid/Iscomatrix/Sub Cut

| | | | | | |
|---|---|---|---|---|---|
| 1 | 11.65 | + | + | 1 | 87.63 |
| 2 | 105.01 | + | + | 1 | 93.06 |
| 3 | 7.9 | + | + | 1 | 81.17 |
| 4 | 17.32 | + | + | 1 | 87.12 |
| 5 | 64.15 | + | + | 1 | 80.39 |
| 6 | 168.25 | + | + | 1 | 97.21 |
| Mean | 62.38 | | | | |
| St Dev | 64.19 | | | | |

B—1 mM PG/Iscomatrix/Intramuscular

| | | | | | |
|---|---|---|---|---|---|
| 1 | 22.89 | + | + | 0 | 95.62 |
| 2 | 23.72 | + | + | 0 | 100.50 |
| 3 | 8.82 | + | + | 0 | 95.02 |
| 4 | 7.63 | + | + | 1 | 90.20 |
| 5 | 21.04 | + | + | 0 | 96.45 |
| 6 | 104.06 | + | + | 0 | 94.34 |
| Mean | 31.26 | | | | |
| St Dev | 36.31 | | | | |

C—F Toxoid/Isomatrix/Sub Cut

| | | | | | |
|---|---|---|---|---|---|
| 1 | 46.38 | + | + | 0 | 92.76 |
| 2 | 49.03 | + | + | 0 | 94.57 |
| 3 | 34.71 | + | + | 0 | 97.53 |
| 4 | 21.32 | + | + | 0 | 90.49 |
| 5 | 129.41 | + | + | 0 | 96.70 |
| 6 | 0 | + | − | 1 | 79.02 |
| Mean | 46.81 | | | | |
| St Dev | 44.30 | | | | |

D—F Toxoid/Isomatrix/Intramuscular

| | | | | | |
|---|---|---|---|---|---|
| 1 | 138.42 | + | + | 0 | 92.86 |
| 2 | 155.14 | + | + | 0 | 97.49 |
| 3 | 78.82 | + | + | 0 | 97.63 |
| 4 | 127.56 | + | + | 0 | 98.95 |
| 5 | 146.67 | + | + | 0 | 97.34 |
| 6 | 186.80 | + | + | 0 | 95.38 |
| Mean | 138.90 | | | | |
| St Dev | 35.66 | | | | |

TABLE 4
The effect of route, adjuvant and toxoid administration
on the survival of mice following exposure to 5 x
LCt₅₀ of aerosolised crude ricin

| | |
|---|---|
| Alhydrogel | Iscomatrix |

| | Subcu- | Intra- | Subcu- | Intra- |
|---|---|---|---|---|
| | taneous | muscular | taneous | muscular |
| Saline | 0 | 0 | 0 | 0 |
| 20 mM PG | 1 | 2 | 0 | 0 |
| Toxoid | | | | |
| 1 mM PG | 3 | 6 | 6 | 5 |
| Toxoid | | | | |
| Formaldehyde | 6 | 6 | 5 | 6 |
| toxoid | | | | |

Body Weight

Measurement of body weight change is a good indicator of animal health and may help in discriminating differences in the quality of protection offered by F and 1 mM PG toxoids. A comparison of body weight between the vaccine groups over the first three days following ricin challenge is shown in FIG. 7. Beyond this time point, differing survival levels made interpretation of the data difficult. Animals vaccinated with saline and the 20 mM PG toxoid responded in a similar manner following toxin exposure, both losing up to 20% of their initial pre-exposure weights. When the data are analysed solely by group (i.e. all 1 mM PG versus all F toxoid animals) animals administered either F or 1 mM PG toxoid lost less weight than controls. However, this was only significant for the F toxoid data set. The failure of the 1 mM PG data set to reach significance was due to the poor effect of this vaccine administered via the subcutaneous route.

There was a significant negative correlation between body weight change and both circulating ricin specific IgG1 antibodies (p<0.001) and survival (p<0.05) (data not shown). However, in all cases, those animals administered vaccine via the subcutaneous route continuously lost weight over the first three days. Those animals administered vaccine via the intramuscular route began to recover more quickly with the average body weight increasing after day 2.

Skins and Symptoms of Toxicity

Although signs and symptoms of ricin intoxication were assessed in all surviving animals for up to 7 days, comparison between groups were made at day 3 when all animals were alive (Table 5). Animals began to show signs of ricin poisoning within 24 hours of exposure and in the control and 20 mM PG toxoid groups, these signs gradually increased in intensity until death.

Although animals receiving F toxoid or 1 mM PG toxoid had significantly fewer signs and symptoms of intoxication the groups that were administered 1 mM PG toxoid tended to have more pronounced signs of intoxication than the F toxoid group. There was a significant (p<0.01) correlation between the signs and symptoms of ricin intoxication and circulating ricin specific antibodies (data not shown), indicating that the lower the antibody concentrations in the blood the poorer the quality of protection.

TABLE 5
The effect of route, adjuvant and toxoid on the signs
and symptoms of ricin intoxication three days following exposure
to 5 x LCt₅₀ of aerosolised crude ricin

| | Alhydrogel ® | | Iscomatrix ™ | |
|---|---|---|---|---|
| | Subcu- | intra- | Subcu- | intra- |
| | taneous | muscular | taneous | muscular |
| Saline | 3.3 ± 0.5 | 3.0 ± 0.0 | 3.7 ± 0.5 | 3.3 ± 0.5 |
| 20 mM PG | 3.0 ± 0.0 | 2.2 ± 1.0 | 3.0 ± 0.0 | 3.2 ± 0.4 |
| 1 mM PG | 3.4 ± 0.4 | 0.0 ± 0.0 | 1.0 ± 0.0 | 0.2 ± 0.4 |
| Formaldehyde | 1.7 ± 0.5 | 0.0 ± 0.0 | 0.2 ± 0.4 | 0.0 ± 0.0 |

Lung Wet Weight

The lungs from all surviving animals culled at day 7 post ricin challenge were weighed and data combined to compare the effect of route, toxoid and adjuvant. There was no statistical difference between lung wet weights for 1 mM PG and F toxoid groups. The effect of route and adjuvant are shown in FIG. 8. Surviving animals vaccinated via the subcutaneous route and formulated with alhydrogel had significantly heavier lungs than animals vaccinated via the intramuscular route or formulated with Iscomatrix. This supports the observation that animals administered vaccine via the subcutaneous route and formulated with alhydrogel are afforded least protection against inhaled ricin. Lung wet weights show a negative correlation with circulating levels of ricin specific IgG1 antibodies (p<0.001) and with the percentage change in body weight 3 days after ricin challenge (p<0.0001). Conversely, a significant positive correlation was observed with signs and symptoms of toxicity (p<0.0001).

Discussion

In this study we have used a previously characterised toxoiding approach (F toxoid formulated with Alhydrogel) to investigate the potential of an alternative toxoiding process, one that utilises phenyl glyoxal, to generate novel ricin vaccine candidates. Immunisation of Balb/C mice with toxoid prepared using 1 mM PG was well tolerated by all animals throughout the immunisation schedule and induced high concentrations of circulating ricin specific IgG1 antibodies which resulted in the majority of animals (20/24) surviving exposure to a supralethal dose of aerosolised toxin. During the following 7 day observation period, these animals exhibited minimal signs and symptoms of intoxication and a change in body weight that was less pronounced than that observed for animals immunised with saline. These observations are comparable to results obtained with F toxoid (23/24 survived) and support our contention that toxoids produced by chemical modification of ricin holotoxin by PG may offer an alternative approach in vaccination studies that prevent injury and death from ricin exposure.

No licensable product that can protect against the lung damaging effects and death following ricin intoxication is available. However, several immunological based strategies are being explored and include the use of ricin specific antitoxins and vaccines. The current vaccine candidates that are in early clinical trials are truncated or mutated forms of ricin A chain. Early studies within our laboratory have consistently found that the protective immune response initiated by A chain candidates was less robust than that offered by formalin based ricin holotoxoid. However, several drawbacks have been identified with formaldehyde based toxoids. These include the incomplete inactivation of toxin and the potential for reversion of toxoid to active toxin. Further, at extended incubation times and at high concentrations of formaldehyde, alterations in the tertiary structure of proteins may develop. These problems would need to be overcome if ricin toxoid was to be seriously considered as a viable alternative to the subunit vaccine approach.

In a novel approach we have explored the utility of toxoiding ricin with PG. Phenyl glyoxal is a reactive ketoaldehyde often used as an investigative tool to study the role of arginine residues in proteins, including ricin A chain. Other toxoiding procedures have been explored for their ability to inactivate toxin without a reduction in immunogenicity but have shown varying degrees of success. Recent work in our laboratories has shown that PG based ricin toxoids, at concentrations above 1 mM, can alter the tertiary structure of the protein. This has a major impact on the immunogenicity of the toxoid as in this study 20 mM PG toxoid did not elicit an immune response and was as ineffective as saline in protecting animals from the lethal effects of inhaled toxin. At 1 mM PG, structural integrity of ricin is maintained whilst the cytotoxic activity of the protein is eliminated. This PG toxoid was also found to be highly immunogenic and able to elicit a protective immune response that was comparable to F (formaldehyde) toxoid.

Death from inhaled ricin emanates from severe pulmonary damage resulting in pulmonary edema and consequential respiratory failure. In this study, lung wet weights (from survivors only taken at day 7) following administration of PG and F toxoid were the same. However, animals administered Iscomatrix via the intramuscular route displayed significantly Lighter lung wet weights, indicating better protection against the damaging effects of ricin. It is generally believed that protection is achieved through the neutralisation of ricin by specific antibodies. Our data support this view and indicate a correlation between higher levels of circulating ricin-specific antibodies and less lung damage, a correlation that is also borne out with the signs and symptoms of intoxication and the percentage drop in body weight (Table 3). Of the signs and symptoms, body weight has long been used by toxicologists as a sensitive index of well being. In this study unprotected animals lost up to one fifth of their initial body weight by the third day following ricin challenge. Animals vaccinated with 20 mM PG toxoid lost similar levels of body weight to the saline control group indicating, along with subsequent deaths, that this was not a protective vaccine. This correlates with the findings of others who showed evidence for major structural alterations in ricin caused by incubation with 20 mM PG. The lack of protection would strongly suggest that the resultant structural changes altered the appropriate antigenicity of the ricin.

Studies have shown that the immune response induced by vaccine administration not only varies from animal to animal but also varies as a function of route. The choice of route must therefore be carefully considered when deciding on the type and magnitude of the response required. The immune cell profile is known to differ from location to location and exposure to exogenous antigens can result in further changes in the patterns of local T and B cells and secreted cytokines. It is the tissue cytokine profile that dictates the Th1/Th2 balance and thus the characteristics of the immune response. In the present study, primary and booster vaccinations were given via the same route with the intramuscular route proving more efficacious. The reason for this is unclear although degradation, presentation and drainage from different compartments are important in the immunological response. Several studies have also shown that changing the route of vaccination during the immunisation protocol can synergistically broaden and improve the immune response. This has not yet been investigated with the PG toxoid, but mucosal immunisation is an additional route that is under consideration, especially since we are most concerned with protection against inhaled ricin.

Effective vaccines against important toxins such as ricin are required. The role of adjuvants in the induction and enhancement of the immune response following vaccination has been known since the first studies of in 1926 where alum was used to enhance the immunological properties of toxoid. Adjuvants can be defined as compounds that bias the immune system toward Th1 or Th2 immunity and significantly enhance the immune response against an antigen. As adjuvants used in human use must fulfil stringent requirements the availability of suitable adjuvants is limited. Currently, aluminium based compounds are the only adjuvants used widely in human and veterinary vaccines and as such they have become the benchmark or reference for evaluating new adjuvant formulations. In the present study formulations of F and 1 mM PG toxoids mixed with alhydrogel were effective at inducing a Th2 response. However, the use of Iscomatrix within the formulation induced a much higher Th2 response for the same dose of antigen with a concomitant mild Th1 response. Iscomatrix is a known potent immunomodulator resulting in an induction of a variety of cytokines which mediate both antibody and cellular immune responses and increases the number of MHC class II positive cells. These properties of Iscomatrix adjuvant may lead to a more efficient generation of T helper cell responses, enhancing the B cell mediated antibody responses.

These results show that phenylglyoxal can be used to chemically toxoid ricin to produce a structurally stable vaccine candidate that can induce a protective immune response. This response is equally as effective as the current formaldehyde based toxoid and if administered via the intramuscular route and formulated with ISCOMATRIX may offer a good approach for the development of a ricin vaccine for human use.

### EXAMPLE 7

Ability of PG Toxoid to Generate Therapeutic Antibodies.

In order to evaluate the immunogenicity of toxoids prepared using the method and dicarbonyl toxoiding reagent of the present inventions, groups of six sheep were immunised with a pharmaceutical composition comprising 100 micrograms of toxoid and Freunds Complete adjuvant and later boosted with the same quantity of toxoid and Freunds Incomplete adjuvant at weeks 6, 12, 18, 24 & 30. Ricin was toxoided with formaldehyde or phenylglyoxal as described above. The results are shown in FIG. 9 as antibody titres as measured from a standard ELISA assay as is known in the art, specific for anti-toxoid antibodies. FIG. 9A shows results obtained from toxoid prepared using traditional formaldehyde inactivation and FIG. 9B shows comparable results from phenylglyoxal inactivation.

The results show that even with vaccination of 100 micrograms of toxoid, toxoid prepared using phenylglyoxal is at least as immunogenic as toxoid prepared using the traditional formaldehyde inactivation. In fact, at week, after 50 week, antibody titres from PG-toxoid reached a maximum of approximately 80 mg/ml whereas maximum antibody titres from formaldehyde toxoid over a similar time period reached a maximum of approximately 45 mg/ml, demonstrating that toxoids prepared with PG have the potential to be at least as good as, if not better than, toxoids prepared with formaldehyde, as they are at least as immunogenic and protective as formaldehyde toxoids and have the additional advantage of being less toxic and enabling complete inactivation of toxins at low concentrations and in very short periods of time, 24 hours or less.

### EXAMPLE 8

Toxoids Prepared from Additional Toxins Which Do Not Cause Cell Death; Cytotoxicity of Cholera Toxin

Diphtheria and cholera toxins were both purchased from Sigma-Aldrich, Dorset, UK. Clostridium difficile (C. difficile) toxin A was purchased from Quadratech Ltd, Epsom, Surrey, and ricin toxin was prepared in house at Dstl from seeds of Ricinus communis var. zanzibariensis in the same way as described in Example 1. All toxins were reconstituted with sterile water to give the following final concentrations:

| | |
|---|---|
| Cholera | 2.0 mg/ml |
| Clostridium difficile toxin A | 1.0 mg/ml |
| Diphtheria | 1.0 mg/ml |
| Ricin | 1.6 mg/ml |

The toxin solutions were divided into 50 μl aliquots and frozen in O-ring sealed Eppendorf™ tubes at −80° C. until use.

Cytotoxicities of diphtheria, ricin and C. difficile toxins in Vero cells were determined as before and results are shown FIG. 10A. It was found that each of these toxins was cytotoxic in Vero cell cultures as demonstrated by the concentration-dependent reduction in Vero cell viability after a 48 hour exposure. Viability was determined by incubating with WST-1 reagent for 3 hours and reading on a plate reader at 450 nm. The concentration causing death of 50% of the susceptible cells (LC₅₀) was calculated using a 4-parameter logistic fit to the data using GraphPad Prism 4.0. These values are displayed in FIG. 10A and are C Diff=7 pM, Ricin=0.9 pM, Diphtheria=0.2 pM

Cholera toxin does not cause cell death and therefore its effects can not be investigated using the same cytotoxicity assay. An alternative assay was used which measured cyclic AMP (adenosine monophosphate) in Vero cells, as this is known to increase when these cells are exposed to cholera toxin. Vero cells were incubated with three concentrations of cholera toxin (10 nM, 20 nM and 40 nM) for 1 hour. Hydrochloric acid was used to lyse the cells and the resulting supernatant was run using a commercial cyclic AMP (cAMP) ELISA kit to measure any changes. The results depicted in FIG. 10 show a dose dependent increase in the cAMP level of the Vero cells after 1 hour incubation with cholera toxin (see FIG. 10B).

### EXAMPLE 9

Ability of Different α-Dicarbonyl Reagents to Produce Toxoids

The ability of the α-dicarbonyl toxoiding reagents of general structure R—C(O)C(O)H to inactivate (toxoid) the toxins described in Example 8 was demonstrated using phenylglyoxal (PG), 2,3 butanedione (BD) and 1,2 cyclohexanedione (CHD) (all purchased from Sigma-Aldrich, Dorset, UK). The dicarbonyl reagents were prepared at concentrations of 0.5 mM for PG and CHD and 5 mM for BD, in a bicarbonate buffer at pH 8.3. To examine their effects on toxin activity, a toxin solution (75 nM final concentration) was incubated with each dicarbonyl reagent for different time periods at 37° C. before assessment of residual toxicity using t he Vero cell viability assay.

PG Toxoiding Results:

The results for PG toxoiding are presented below. FIG. 11A shows that exposure to 0.5 mM PG for 24 hrs at 37° C. completely inactivates ricin and diphtheria toxins but not the C. difficile toxin A. This toxin was only fully toxoided under these conditions after a 7 day incubation as shown in FIG. 11B. Cholera toxin also appeared to be partially inactivated under these conditions as the cAMP levels in Vero cells are greatly reduced after a 1 hour exposure to 40 nM of cholera toxin toxoided for 7 days with 0.5 mM PG, in comparison to cells that had a 1 hr exposure to 40 nM of cholera toxin only (as shown in FIG. 11C). Other toxoiding time points were not investigated with this toxin. This demonstrates that at a concentration of 0.5 mM, PG is an effective toxoiding agent and has significant advantages over the traditional formaldehyde treatment as it acts much faster, toxoiding after just 24 hrs with ricin and diphtheria toxins as opposed to the 3-4 week exposure required by formaldehyde.

CHD and BD Toxoiding Results

Exposure to 0.5 mM CHD for 7 days at 37° C. inactivated cholera toxin (FIG. 12) but did not inactivate ricin or diphtheria toxin (C. difficile was not tested) (see FIG. 2.5). Exposure to BD at 5 mM for 7 days (37° C.) inactivated diphtheria and cholera toxins and reduced the toxicity of ricin but did not fully inactivate it (C. difficile was not tested). These data show that PG is much more effective as a toxoiding agent than these two other dicarbonyl toxoiding reagents.

### EXAMPLE 10

Effect of Temperature on Toxoiding Ability of Phenylglyoxal (PG)

The effect of temperature on the ability of PG to toxoid ricin toxin was assessed. All previous experiments were performed with an incubation temperature of 37° C. which was shown to inactivate ricin in just 24 hours. An experiment was conducted to investigate whether PG will toxoid at room temperature (around 20° C.). PG (0.5 mM) in bicarbonate buffer at pH 8.3 was incubated with a 75 nM solution of ricin toxin for either 24 hours or for 7 days at room temperature. The residual activity of the PG-treated ricin was assessed using the Vero cell viability assay. It was found that after a 24 hour exposure to 0.5 mM PG at room temperature the toxicity of ricin has been reduced by 1000 fold as shown by FIG. 14. However a 7 day treatment under the same conditions is seen to fully toxoid ricin. It is quite feasible that a higher concentration of PG could be employed which would toxoid more quickly (in less than than 7 days) whilst preserving immunogenicity. This is another advantage of PG over the traditional formaldehyde treatment which requires a temperature of 37° C. to inactivate toxin.

### EXAMPLE 11

Effect of pH and Storage Temperature on the Stability of PG/Ricin Toxoid

The stability of the PG-toxoided ricin was assessed over time at two different storage temperatures of −20C. and 4° C. and in three storage buffers. Ricin was toxoided with 0.5 mM PG in bicarbonate buffer pH 8.3 at 37° C. for 7 days. The excess PG was then removed using a NAPS 10 column and the toxoided toxin eluted with either a bicarbonate buffer at pH 8, sodium acetate-acetic acid buffer at pH 5 or with PBS at pH 7. Cytotoxicity was assessed using the Vero cell viability assay at 1, 2, 3 and 6 months after storage of these samples at either 4° C. or −20° C. Table 6 displays the data. The results show that the toxoid is extremely stable at both 4° C. and −20° C. in all 3 storage buffers and demonstrates that the toxoid does not require an excess of PG to be present in the storage buffer to maintain stability. This is a significant advantage over formaldehyde toxoids where an excess of formaldehyde is required to prevent reversion. Stability at 4° C. is also a benefit as th is makes storage and transport of PG toxoids easier.

TABLE 6
LC₅₀ of ricin toxoided with 0.5 mM PG and stored at
various temperature and in various storage buffers

| Storage | Storage | LC₅₀ (pM) | | | |
|---|---|---|---|---|---|
| Temp ° C. | Buffer | 1 month | 2 months | 3 months | 6 months |
| 4 | pH 8 | >3750 | >3750 | >3750 | >3750 |
| 4 | pH 7 | >3750 | >3750 | >3750 | >3750 |
| 4 | pH 5 | >3750 | >3750 | >3750 | >3750 |
| −20 | pH 8 | >3750 | >3750 | >3750 | >3750 |
| −20 | pH 7 | >3750 | >3750 | >3750 | >3750 |
| −20 | pH 5 | >3750 | >3750 | >3750 | >3750 |

### EXAMPLE 12

Quality of Protection Achieved Using a PG Toxoid in an in Vivo Model

The ability of a PG toxoid to elicit a protective immune response in vivo was investigated by examining the immunogenicity and the protective efficacy of a PG C. difficile toxoid in Balb/C mice exposed to 3 LD₅₀s of C. difficile toxin A. The quality of protection was monitored by measuring survival, body weight change and the signs and symptoms of C. difficile intoxication for up to 7 days post toxin challenge. The level and neutralising ability of specific antibody against the toxoid was assessed in vitro.

Female Balb/C mice (Charles-River Laboratories, Margate, Kent, UK: 20 g bodyweight) were divided into four groups (n=6 animals/group) consisting of vehicle control (PG only) low dose (625 ng toxoid), high dose (2.5 μg toxoid) and positive control (toxin only). C. difficile toxin A was toxoided with 0.5 mM PG in bicarbonate buffer at pH 8.3 for 7 days at 37° C. Mice were injected intramuscularly with 50 μl of toxoid solution on days 0 and 42. On day 63 all animals were exposed to 3LD₅₀s (30 ng) of C. difficile toxin A injected by the intraperitoneal route. The animals were culled 14 days after challenge and blood collected by cardiac puncture to measure specific antibody level by ELISA.

The results show that the control animals that were not immunised with the toxoid died 48 hours after challenge with 3LD₅₀s of toxin. The low dose group showed signs of poisoning after 24 hours, displaying ruffled fur and hunched posture, but all made a full recovery by 48 hours. The high dose group showed no signs of poisoning and all survived the toxin challenge. The results of the ELISA for specific antibody against C. difficile toxin are shown in FIG. 15. It can be seen that there are high levels of antibody present in the high dose animals (FIG. 15A) in comparison to the naive control animals. The amount of antibody present in the low dose group is much lower and more variable between individual animals (FIG. 15B). These data prove that the PG toxoid is immunogenic and the antibodies raised provide full protection against a lethal toxin challenge.

An in vitro neutralising assay was then performed using the serum from animal HD1 as it was seen by ELISA to have the highest level of specific antibody to C. difficile toxin. The serum was initially diluted 1 in 5 with medium and then serially diluted 1:1 to give increasing dilutions of serum. This was then incubated for 1 hour on the bench with a final concentration of C. difficile toxin equivalent to ˜3×LC₅₀ (25 pM) in tissue culture medium. Following this incubation 100 μl of this mixture was added to the Vero cells for 48 hours at 37° C. 5% CO ₂ and cell viability assessed with WST-1 reagent as before. The results are shown in FIG. 16.

It can be seen that at a high concentration of serum the cytotoxicity of C. difficile toxin is fully inhibited presumably by neutralising antibodies present in the serum. As the concentration of serum decreases the neutralising activity diminishes and cell viability decreases. These results confirm the in vivo data demonstrating that the antibodies raised by the PG toxoid are protective.

### EXAMPLE 13

Circular Dichroism Spectroscopy of Toxoids

Formaldehyde toxoid was prepared following the addition of formaldehyde (37% v/v) to ricin (1 mg/ml) to give a final concentration of 2.5% v/v. This mixture was incubated at 37° C. for 3 weeks after which lysine was added to a final concentration of 0.1M. The solution was then desalted into saline using PD10 chromatography columns. Phenylglyoxal (PG) toxoid was prepared by the addition of equal volumes of toxin (2 mg/ml) and PG (0 to 40 mM in bicarbonate buffer, pH 8) to give a final concentration of 1 mg/ml toxin in 0 to 20 mM PG. The solutions were then incubated either at room temperature for 24 and 96 hours or for 24 hours at 37° C. After incubation, the PG toxoid was desalted into saline using PD10 chromatography columns. Toxoid concentrations were measured using a previously published method (Griffiths et al., 1999, supra) and stored frozen at −80° C. until required. PG toxoids with a PG concentrations of 0, 0.001, 0.01, 0.05, 0.1, 0.5, 1.0, 2.5, 5.0 and 20 mM. A single formylated ricin sample (300 mM) was studied

PG toxoids incubated at RTP for 24 hr & 96 hr (0-20 mM) and at 37° C. for 24 hr (0-20 mM) were allowed to thaw naturally at room temperature prior to the experiments. All samples were diluted 1:1 ratio with 10 mM phosphate buffer, pH7.0 and measured in 10 mm & 0.5 mm cell pathlengths. UV and CD spectra were measured with the Applied Photophysics Ltd Chirascan spectrometer in the regions 400-230 nm & 260-190 nm. The following parameters were applied: 1 nm spectral bandwidth, 0.5 nm stepsize, 1.5 s (400-230 nm) and 3.0 s (260-190 nm) time-per-point. All spectra were baseline corrected and where appropriate temperature was recorded. The far-UV CD spectra were corrected for concentration and pathlength and are expressed in terms of mean residue molar ellipticity, [θ], or mean residue molar extinction coefficient, Δε(M⁻¹ cm⁻¹) (mean residue molecular weight 113 was used).

The CD spectrum of each solution was recorded at room temperature (20° C.), heated to high temperature (90° C.) and re-cooled to 20° C. after heating. Melting profiles were also monitored at λ=233 nm. The instrument was equipped with a Melcor Thermoelectric Peltier unit set to change temperature from 20-96° C. at a rate of 1° C./min with a 1° C. stepsize, and a 0.2° tolerance. A 10 s time-per-point CD measurement time was employed. Temperature was measured directly with a thermocouple probe in the protein solution. All measurements were done in a rectangular 0.5 mm cell pathlength. CD versus temperature plots was fitted to a 1-component Van't Hoff equation using the Origin software (Origin Lab Corporation, USA).

pH titrations were measured using a Corning pH105 pH meter with a ThermoRussel K series electrode. Aliquots of NaOH and HCl were used to change the pH in the range pH2-8. Theoretical pH curves were produced in the Origin v7.5 software.

An overlay of the far-UV CD spectra of whole native ricin, the 1 mM PG ricin toxoid and the formylated ricin toxoid is presented in FIG. 17. 1 mM PG clearly has a minimal effect on the CD spectrum and importantly the 223 nm positive feature is largely maintained inferring little or no change in ricin conformation. On the other hand, formylation leads to the loss of the 233 nm positive feature and an overall reduction in CD spectrum indicating a reduction in ordered conformation (unfolding).

The far-UV CD spectra monitoring the effects of different PG levels with various incubation conditions are illustrated in FIG. 18. Irrespective of the incubation conditions, higher PG levels result in CD spectra changes and hence protein conformation. As a function of PG concentration, incubation at room temperature for both 24 & 96 hr RTP sees a progressive decrease in CD signal with the noticeable loss of the 233 nm positive CD feature associated with the B-chain. An apparent “isobestic point” is observed at ˜225 nm indicative of a two component denaturation of the B-chain. Incubation at 37° C. RTP with higher PG levels (5 mM and 20 mM PG) indicates further denaturation. A plot of CD_{233 nm} (B-chain integrity) versus phenylglyoxal content is presented in FIG. 18D which graphically illustrates the denaturation process.

During measurements made as part of the current work, it was noticed that solutions of PG toxoids gave a pronounce absorbance at 250 nm related to the “concentration” of PG in the various toxoids (data not presented). This prevents the use of A₂₈₀ as a measure of protein concentration.

Formylation is known to occur at lysine residues; the ricin A-chain contains only 2 lysine residues but ricin B-chain contains 7, hence the greater sensitivity of the ricin B-chain to formylation. Viewing the amino acid sequence and X-ray structure structure using the SwissProt PDB viewer revealed that 5 of the ricin B-chain lysine residues (K40, K62, K168 & K203) are spatially close to a disulphide bond. Therefore, it is not surprising that formylation of the ricin B-chain perturbed the disulphide conformation responsible for the 233 nm positive CD feature.

Phenylglyoxalation is known to occur at arginine residues. There are 21 arginine residues in the ricin A-chain and 13 arginine residues in the ricin B-chain distributed evenly throughout both domains. The CD results presented here indicate that only at higher phenylglyoxalation (>1 mM) is there a considerable effect on secondary structure. Coupled with the toxicity results, the CD data indicate that the inhibition by low levels of phenylglyoxal (modification of arginine) is not associated with a secondary structure change. On the other hand, formylation induced secondary structure changes.

From the above examples it may be concluded the present invention is particularly advantageous in that, α-dicarbonyl toxoiding reagents of the invention, such as PG, are particularly effective toxoiding reagents which provide the same toxoiding capability as formaldehyde but which have the ability to be used at lower temperatures, for shorter periods of time and at lower concentrations to produce stable toxoids which do not need to be formulated with any additional amounts of the toxoiding agent. For example, PG at a concentration of 0.5 mM toxoids ricin, diphtheria, cholera and C. difficile toxins. Toxoiding is achieved at 37° C. in 7 days or less. At 20° C. PG will inactivate ricin in 7 days or less. Other related glyoxals also produce inhibition of toxin activity but do not appear to be as widely applicable as PG. The PG toxoid of ricin toxin is stable for at least six months at pH 5, 7 and 8 without a need for excess PG to be present in the preparation. The PG toxoid of C. difficile toxin elicits a response capable of protecting mice against a 3 LD₅₀ challenge with C. difficile toxin. This toxoid induces the production of antibodies against C. difficile and these antibodies neutralise toxin in vitro as demonstrated by the prevention of cytotoxicity in Vero cells.

1. A toxoid derived from a toxin in which an arginine residue within the toxin has undergone reaction with an α-dicarbonyl compound of general structure RC(O)C(O)R′, for use as a medicament.

2. A toxoid according to claim 1 wherein the α-dicarbonyl compound is selected from the group consisting of glyoxal, methylglyoxal, butanedione, 1,2-cyclohexanedione, phenylglyoxal, 4-fluorophenylglyoxal, 4-nitrophenylglyoxal and 4-hydroxyphenylglyoxal.

3. A toxoid according to claim 2 wherein the α-dicarbonyl compound is phenylglyoxal.

4. A toxoid according to claim 1 which is derived from a plant toxin.

5. A protein toxoid according to claim 4 wherein the plant toxin is ricin.

6. A toxoid according to claim 1 wherein the toxin is derived from an animal.

7. A toxoid according to claim 6 wherein the toxin is a snake toxin.

8. A toxoid according to claim 1 wherein toxin is derived from a bacterium.

9. A toxoid according to claim 8 wherein the toxin is botulinum toxin, tetanus toxin, diphtheria toxin, cholera toxin, Bordetella pertussis toxin, pseudomonas endotoxin, shiga toxin or shiga like toxin, anthrax or SEB.

10. A toxoid according to claim 1 in which the medicament is a vaccine.

11. A toxoid derived from a toxin which has undergone reaction with an α-dicarbonyl compound of general structure R—C(O)C(O)H, for use in the prophylactic or therapeutic treatment of intoxication by the toxin.

12. A toxoid according to claim 11 wherein the α-dicarbonyl compound is selected from the group consisting of glyoxal, methylglyoxal, butanedione, 1,2-cyclohexanedione, phenylglyoxal, 4-fluorophenylglyoxal, 4-nitrophenylglyoxal and 4-hydroxyphenylglyoxal.

13. A toxoid according to claim 12 wherein the α-dicarbonyl compound is phenylglyoxal.

14. A toxoid according to claim 11 wherein the α-dicarbonyl compound is in solution at a concentration of from about 0.05 mM to about 5 mM.

15. A toxoid according to claim 14 wherein the concentration of α-dicarbonyl compound is approximately 1 mM.

16. A toxoid according to claim 11, wherein the dicarbonyl compound is in solution buffered to a pH in the range of from 6 to 14.

17. A toxoid according to claim 16 wherein the pH is 8.

18. A toxoid according to claim 11 wherein the reaction is conducted at 37° C. for at least 1 hour.

19. A toxoid according to claim 11 wherein the reaction is conducted at 37° C. for between 1 and 168 hours.

20. A toxoid according to claim 19 wherein the reaction is conducted at 37° C. for approximately 24 hours.

21. A toxoid according to claim 11 wherein the toxin is derived from a plant.

22. A toxoid according to claim 21 wherein the toxin is ricin.

23. A toxoid according to claim 11 wherein the toxin is derived from an animal.

24. A toxoid according to claim 23 wherein the toxin is a snake toxin.

25. A toxoid according to claim 11 wherein toxin is derived from a bacterium.

26. A toxoid according to claim 25 wherein the toxin is a botulinum toxin, tetanus toxin, diphtheria toxin, cholera toxin, Bordetella pertussis toxin, pseudomonas endotoxin, shiga toxin or shiga like toxin, anthrax or SEB.

27. A pharmaceutical composition comprising the toxoid according to claim 1, together with a pharmaceutically acceptable diluent or carrier.

28. A pharmaceutical composition according to claim 27, which further comprises an adjuvant.

29. A method of producing an antitoxin which comprises administering to an animal a toxoid or a pharmaceutical composition according to claim 1 in an effective amount so as to induce production of anti-toxoid antibodies and taking blood from said animal, separating serum from the blood and extracting antibodies from the serum.

30. A method according to claim 29 wherein the extracted antibodies are fragmented to produced despeciated antitoxin antibody fragments.

31. An antitoxin produced by the method according to claim 29.

32. An antitoxin according to claim 31 for use in the treatment of toxin intoxication.

33. A method of treating an intoxicated individual comprising administering thereto a therapeutically effective amount of an antitoxin produced according to claim 31.

34. A method of vaccinating against intoxication by a toxin, comprising administering to a mammal a pharmaceutically effective amount of a toxoid according to claim 1.

35. A method of treating an intoxicated mammal, including man, comprising administering to the mammal a pharmaceutically effective amount of the antitoxin according to claim 31.

36. A method of producing an antitoxin which comprises administering to an animal a toxoid or a pharmaceutical composition according to claim 11 in an effective amount so as to induce production of anti-toxoid antibodies and taking blood from said animal, separating serum from the blood and extracting antibodies from the serum.
